(19) United States
(12) Patent Application Publication
(10) Pub. No.: US 2025/0037796 A1
(43) Pub. Date: Jan. 30, 2025

(54) METHODS FOR DETECTING ABSENCE OF HETEROZYGOSITY BY LOW-PASS GENOME SEQUENCING

(71) Applicant: The Chinese University of Hong Kong, Hong Kong (CN)

(72) Inventors: Kwongwai CHOY, Hong Kong (CN); Zirui Dong, Shenzhen (CN); Ye Cao, Jiangxi (CN); Zhenjun Yang, Shenzhen (CN)

(21) Appl. No.: 18/778,481

(22) Filed: Jul. 19, 2024

Related U.S. Application Data
(63) Continuation of application No. 17/005,569, filed on Aug. 28, 2020, now abandoned.
(60) Provisional application No. 62/894,497, filed on Aug. 30, 2019.

(51) Int. Cl. G16B 30/10; C12Q 1/6869; G16B 20/10; G16B 20/20

(52) U.S. Cl. CPC G16B 30/10; C12Q 1/6869; G16B 20/10; G16B 20/20

(57) ABSTRACT

The present application provides methods of detecting absence of heterozygosity (AOH) in a biological sample from a subject, and computer readable mediums and devices for carrying out the methods.

### CROSS-REFERENCE TO RELATED APPLICATION

This present application is a continuation application to U.S. patent application Ser. No. 17/005,569, filed on Aug. 28, 2020, which claims priority to U.S. Provisional Patent Application No. 62/894,497 filed on Aug. 30, 2019, the contents of which are incorporated herein by reference in its

### FIELD OF THE INVENTION

The present application generally relates to the field of molecular genetics and molecular biology. In particular, the present application provides methods and tools for detecting absence of heterozygosity (AOH) in a subject.

### BACKGROUND

Absence of heterozygosity (AOH) is one of the genomic changes that causes human diseases including congenital disorders [1, 2] and tumor oncogenesis [3, 4] as a result of the absence of wild-type or imprinted genomic sequences. Apart from a heterozygous deletion event, AOH is commonly presenting as a copy-number neutral event, potentially representing runs of homozygosity or long contiguous stretch of homozygosity [5] and evidence for identity-by-descent (such as parental consanguinity) or uniparental disomy (UPD) [6]. The prevalence of human diseases caused by UPD is estimated to be 1 in 5,000 of livebirths [7], results when UPD involves chromosomes (chromosomes 6, 7, 11, 14, 15 or 20) associated with imprinting [8]. For instance, ˜25% of cases with Prader-Willi syndrome (OMIM #: 176270) result from maternal UPD of chromosome 15 [9, 10] due to AOH or uniparental heterodisomy, where both alleles of the same chromosomal region are inherited from one parent.

In the routine clinical setting, chromosomal microarray analysis (CMA) with single nucleotide polymorphism (SNP) probes is the gold standard for identification of AOH at a resolution of >5-Mb [5, 6]. Currently, owing to the breakthrough of molecular technologies such as next-generation sequencing over the years, exome sequencing (ES) has been utilized for clinical diagnostic testing [11-16] and researchers have begun to investigate AOH by using the detection of single nucleotide variants (SNVs) [17, 18]. Compared with genome sequencing (GS), ES shows limited ability in detection of copy-number variants (CNVs) and even SNVs due the capture biases [6, 19]. However, despite the advantages of GS, current clinically available approaches are based on low-pass (low-coverage) GS with a read-depth ranging from ˜0.1 to >5-fold due to the affordable cost. Recent studies have demonstrated that low-pass GS is able to identify CNVs [20-22] and chromosomal structural rearrangements [23-25] but detection of AOH is not available from current analytic methods. Moreover, uniparental heterodisomy is also cryptic to current low-pass GS.

New methods for detection of AOH particularly by utilizing low-pass GS are needed in the art.

### SUMMARY

In a first aspect, there is provided in the present application a method of detecting absence of heterozygosity (AOH), e.g. copy-number neutral loss of heterozygosity (CN-LOH), in a biological sample from a subject, comprising
(i) receiving sequence reads from low-pass genome sequencing of genomic DNA of the biological sample;
(ii) aligning the sequence reads to a human genome reference, and selecting and sorting sequence reads aligned to the human genome reference based on the aligned chromosome and genomic coordinates;
(iii) identifying single-nucleotide variants (SNVs) in the aligned sequence reads, wherein a single-nucleotide variant at each site has a mutant base type different from the base type at the corresponding site from the human genome reference;
(iv) identifying homozygous SNVs, diploid heterozygous SNVs, or non-diploid heterozygous SNVs from the SNVs identified in step (iii), wherein
   a homozygous SNV is define based on the percentage of sequence reads supporting the mutant base type different from the base type at the corresponding site from the human genome reference being 100%,
   a diploid heterozygous SNV is define based on the percentage of sequence reads supporting the mutant base type different from the base type at the corresponding site from the human genome reference being no less than 25% and no large than 75%,
   a non-diploid heterozygous SNV is define based on the percentage of sequence reads supporting the mutant base type different from the base type at the corresponding site from the human genome reference being less than 25% and larger than 0% or larger than 75% and less than 100%;
(v) determining a rate of homozygous SNVs, diploid heterozygous SNVs, or non-diploid heterozygous SNVs identified in step (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) comparing the rate of homozygous SNVs, diploid heterozygous SNVs, or non-diploid heterozygous SNVs for individual windows determined from step (v) with an average rate of homozygous SNVs, diploid heterozygous SNVs, or non-diploid heterozygous SNVs for corresponding individual windows established from a control population.

In a second aspect, there is provided in the present application a computer system for detecting absence of heterozygosity (AOH), e.g. copy-number neutral loss of heterozygosity (CN-LOH), in a biological sample from a subject, comprising a processor and a memory storing a plurality of instructions, wherein the processor, upon processing the instructions, is configured to:
(i) receive sequence reads from low-pass genome sequencing of genomic DNA of the biological sample;
(ii) align the sequence reads to a human genome reference, and selecting and sorting sequence reads aligned to the human genome reference based on the aligned chromosome and genomic coordinates;
(iii) identify single-nucleotide variants (SNVs) in the aligned sequence reads, wherein a single-nucleotide variant at each site has a mutant base type different from the base type at the corresponding site from the human genome reference;
(iv) identify homozygous SNVs, diploid heterozygous SNVs, or non-diploid heterozygous SNVs from the SNVs identified in (iii), wherein
   a homozygous SNV is define based on the percentage of sequence reads supporting the mutant base type different from the base type at the corresponding site from the human genome reference being 100%,
   a diploid heterozygous SNV is define based on the percentage of sequence reads supporting the mutant base type different from the base type at the corresponding site from the human genome reference being no less than 25% and no large than 75%,
   a non-diploid heterozygous SNV is define based on the percentage of sequence reads supporting the mutant base type different from the base type at the corresponding site from the human genome reference being less than 25% and larger than 0% or larger than 75% and less than 100%;
(v) determine a rate of homozygous SNVs, diploid heterozygous SNVs, or non-diploid heterozygous SNVs identified in (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) compare the rate of homozygous SNVs, diploid heterozygous SNVs, or non-diploid heterozygous SNVs for individual windows determined from (v) with an average rate of homozygous SNVs, diploid heterozygous SNVs, or non-diploid heterozygous SNVs for corresponding individual windows established from a control population.

In a third aspect, there is provided in the present application a computer readable medium storing a plurality of instructions, wherein the plurality of instructions, upon executed by one or more processors, perform an operation including
(i) receiving sequence reads from low-pass genome sequencing of genomic DNA of a biological sample from a subject;
(ii) aligning the sequence reads to a human genome reference, and selecting and sorting sequence reads aligned to the human genome reference based on the aligned chromosome and genomic coordinates;
(iii) identifying single-nucleotide variants (SNVs) in the aligned sequence reads, wherein a single-nucleotide variant at each site has a mutant base type different from the base type at the corresponding site from the human genome reference;
(iv) identifying homozygous SNVs, diploid heterozygous SNVs, or non-diploid heterozygous SNVs from the SNVs identified in (iii), wherein
   a homozygous SNV is define based on the percentage of sequence reads supporting the mutant base type different from the base type at the corresponding site from the human genome reference being 100%,
   a diploid heterozygous SNV is define based on the percentage of sequence reads supporting the mutant base type different from the base type at the corresponding site from the human genome reference being no less than 25% and no large than 75%,
   a non-diploid heterozygous SNV is define based on the percentage of sequence reads supporting the mutant base type different from the base type at the corresponding site from the human genome reference being less than 25% and larger than 0% or larger than 75% and less than 100%;
(v) determining a rate of homozygous SNVs, diploid heterozygous SNVs, or non-diploid heterozygous SNVs identified in (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) comparing the rate of homozygous SNVs, diploid heterozygous SNVs, or non-diploid heterozygous SNVs for individual windows determined from (v) with an average rate of homozygous SNVs, diploid heterozygous SNVs, or non-diploid heterozygous SNVs for corresponding individual windows established from a control population.

In a fourth aspect, there is provided in the present application a device comprising one or more processors and a computer readable medium of the third aspect.

### BRIEF DESCRIPTION OF THE DRAWINGS

FIG. 1A shows the workflow of a method of detecting Absence of Heterozygosity (AOH) according an exemplary embodiment of the present application.

FIG. 1B shows information for each step in FIG. 1A.

FIGS. 2A-2F show correlations of different parameters between GS (30-fold, hereafter referred as GS) and Low-pass GS (low-pass GS, ˜4-fold, hereafter referred as low-pass GS) in sample HG00514. FIG. 2A shows the correlation between the parental genomic differences (Y axis) and the rates of heterozygous SNVs (X axis) in GS data. FIG. 2B shows the correlation between the parental genomic differences (Y axis) and the rates of homozygous SNVs (X axis) in GS data. FIG. 2C shows the correlation between the rates of homozygous SNVs (Y axis) and the rates of diploid heterozygous SNVs (X axis) in GS data. FIG. 2D shows the correlation between the rates of diploid heterozygous SNVs (Y axis) indicated by low-pass GS and the rates of diploid heterozygous SNVs (X axis) calculated by GS data. FIG. 2E shows the correlation between the rates of homozygous SNVs (Y axis) indicated by low-pass GS and the rates of homozygous SNVs (X axis) calculated by GS data. FIG. 2F shows the correlation between the rates of homozygous SNVs (Y axis) and the rates of diploid heterozygous SNVs (X axis) in low-pass GS data. In each figure, P value of Pearson correlation coefficient is shown in red.

FIGS. 3A-3D show accuracy of AOH detection. FIG. 3A shows the consistent of AOH detection between GS and low-pass GS and FIG. 3B shows the sensitivity and specificity of detecting AOH by low-pass GS using the detection results from GS when incorporating the increased rates of homozygous SNVs with decreased rates of heterozygous SNVs. 100% sensitivity and specificity are observed at a resolution of 1.4-Mb. FIG. 3C shows the consistency of AOH detection for five cases in two independent experiments both with low-pass GS and FIG. 3D shows the sensitivity and specificity of detecting AOH in data from 2^{nd} batch of these five samples by using the data from 1^{st} batch as reference. In each figure, the X axis represents the size of AOH detected. The Y axis in FIGS. 3A and 3C indicates the number of AOH detected, while in FIGS. 3B and 3D reflects the sensitivity and specificity for setting different cutoffs of detection resolutions.

FIGS. 4A-4F show detection of AOH in chromosome 5 of sample HG00733. FIG. 4A shows distribution of copy-numbers among the windows (indicated by black dots) in chromosome 5 in this sample. The only deletion is shown by a purple arrow: The X axis shows the genomic location in all figures, while the Y axis indicates the copy-number in FIG. 4A. Distribution of normalized rates of heterozygous SNVs (FIG. 4B) and normalized rates of homozygous SNVs (FIG. 4C) cross chromosome 5 by low-pass GS. Distribution of rates of heterozygous SNVs (FIG. 4E) and rates of homozygous SNVs (FIG. 4F) cross chromosome 5 by GS. In FIGS. 4B and 4D, AOH (indicated by red arrows with the numbers of windows involved in the bottom) identified by observation of consecutive decreased rates of heterozygous SNVs. In FIGS. 4C and 4E, regions with consecutive increased rates of homozygous SNVs (indicated by blue arrows with the numbers of windows involved in the bottom).

FIG. 4F shows distribution of parental genomic differences across chromosome 5. Y axis in FIGS. 4B-4F shows the rate of each corresponding parameter. Genomic region of the large AOH seq[GRCh37] 5q23q34 (149200000_164900000)x2 hmz is shown by a pair of green dotted lines across each figure.

FIGS. 5A-5G show AOH detected in sample 18C1564. FIG. 5A shows copy-number distribution and FIG. 5B shows genotype distribution reported by CMA. The X axis indicates the genomic location in FIGS. 5A and 5B. The Y axis in FIG. 5A shows the log 2 ratio of copy-number, while the Y axis in FIG. 5B shows the distribution of different number of genotypes: 0, 1, 2 and 3 indicates the genotype as A allele, AB, B and AAB/ABB, respectively. In FIG. 5A, each dot represents a probe, the copy-ratio classified as gain, neutral or loss is shown in blue, black and red, respectively. In FIG. 5B, the presence of each type of genotype is shown as a green dot in the corresponding line and the regions with AOH reported are highlighted in green background. Two additional AOH reported by low-pass GS but not by CMA are highlighted in yellow background and the absences of heterozygous genotype (AB) in these two regions are indicated by two red arrows. FIG. 5C shows copy-number distribution reported by low-pass GS with windows indicated by black dots. X axis in FIGS. 5C-5G indicates the genomic locations across chromosome 6, while in FIG. 5C, Y axis represents the copy-number. FIGS. 5D-5F show the distribution of rates of “germline” heterozygous SNVs (AB), homozygous SNVs and “mosaic” heterozygous SNVs (AAB/ABB), respectively. In FIG. 5D, the candidate regions with AOH detected are indicated by each pair of red arrow and the number of windows, while in FIG. 5E, the windows with increased rate of homozygous SNVs within those regions reported in FIG. 5D are shown by each pair of blue arrow and the number of windows. The two cryptic regions only reported by low-pass GS highlighted in FIG. 5B are also highlighted in FIGS. 5D and 5E. In FIG. 5F, the candidate regions with increased rate of “mosaic” heterozygous SNVs are shown by each pair of a blue arrow and the number of windows. In FIG. 5G, Y axis shows maternally inherited genotype in the upper line (in black dots) and paternally inherited genotype in the bottom line (in black dots). The middle line shows in red if the rates of maternal/paternal genotypes are larger than 5 and in blue if the rates are smaller than 0.2.

FIGS. 6A-6G show AOH detected within a mosaic trisomy event in sample 18C1493. FIG. 6A shows copy-number distribution and FIG. 6B shows genotype distribution reported by CMA. X axis indicates the genomic location in FIGS. 6A and 6B. Y axis in FIG. 6A shows the log 2 ratio of copy-number, while Y axis in FIG. 6B shows the distribution of different number of genotypes: 0, 1, 2 and 3 indicates the genotype as A allele, AB, B and AAB/ABB, respectively. In FIG. 6A, each dot represents a probe, the copy-ratio classified as gain, neutral or loss is shown in blue, black and red, respectively. It shows an approximately 40% increase of the whole chromosome 6 (indicated by a blue box). In FIG. 6B, the presence of each type of genotype is shown as a green dot in the corresponding line and the regions with AOH reported are highlighted in green background. FIG. 6C shows copy-number distribution reported by low-pass GS with windows indicated by black dots. The results confirmed approximately 40% increase of the whole chromosome 6 (indicated by a blue line). X axis in FIGS. 6C-6G indicates the genomic locations across chromosome 6, while in FIG. 6C, Y axis represents the copy-number. FIGS. 6D to 6F show the distribution of rates of “germline” heterozygous SNVs (AB), homozygous SNVs and “mosaic” heterozygous SNVs (AAB/ABB), respectively. In FIG. 6D, the candidate regions with AOH detected are indicated by each pair of red arrow and the number of windows, while in FIG. 6E, the windows with increased rate of homozygous SNVs within those regions reported in FIG. 6D are shown by each pair of blue arrow and the number of windows. In FIG. 6F, the candidate regions with increased rate of “mosaic” heterozygous SNVs are shown by each pair of a blue arrow and the number of windows. In FIG. 6G, Y axis shows maternally inherited genotype in the upper line (in black dots) and paternally inherited genotype in the bottom line (in black dots). The middle line shows in red if the rates of maternal/paternal genotypes are larger than 5 and in blue if the rates are smaller than 0.2.

FIGS. 7A-7F show cryptic AOH reported by Low-pass GS. FIGS. 7A, 7C and 7E show the copy-number distribution in 17C1122, 17C1175 and 17C1176, respectively, while FIGS. 7B, 7D and 7F show the distribution of rates of heterozygous SNVs in each of these three samples. X axis in each figure indicate the genomic location. Y axis in FIGS. 7A, 7C and 7E indicate the copy-number while in FIGS. 7B, 7D and 7F show the rate of heterozygous SNVs. In FIGS. 7B, 7D and 7F, the candidate regions with AOH reported by low-pass GS are indicated by each pair of a red arrow and the number of windows involved in the bottom. The green dotted line shows the region of KCTD7 gene across each figure, while in FIG. 7B, the cryptic AOH is highlighted in yellow background.

FIGS. 8A-8F show correlations of different parameters between GS and Low-pass GS in sample HG00733. FIG. 8A shows the correlation between the parental genomic differences (Y axis) and the rates of heterozygous SNVs (X axis) in GS data. FIG. 8B shows the correlation between the parental genomic differences (Y axis) and the rates of homozygous SNVs (X axis) in GS data. FIG. 8C shows the correlation between the rates of homozygous SNVs (Y axis) and the rates of heterozygous SNVs (X axis) in GS data. FIG. 8D shows the correlation between the rates of heterozygous SNVs (Y axis) indicated by low-pass GS and the rates of heterozygous SNVs (X axis) calculated by GS data. FIG. 8E shows the correlation between the rates of homozygous SNVs (Y axis) indicated by low-pass GS and the rates of homozygous SNVs (X axis) calculated by GS data. FIG. 8F shows the correlation between the rates of homozygous SNVs (Y axis) and the rates of heterozygous SNVs (X axis) in low-pass GS data. In each figure, P value of Pearson correlation coefficient is shown in red.

FIGS. 9A-9C show observation of decreased rates of heterozygous SNVs in the region with a heterozygous deletion. Heterozygous deletion arr[GRCh37] 1q23.1q25.2 (158043081_176445395)x1 dn was reported in sample 18C0241. FIG. 9A shows distribution of copy-numbers among the windows (indicated by black dots) in chromosome 5 in this sample. The X axis shows the genomic location in all figures, while the Y axis indicates the copy-number in FIG. 9A. The large deletion is shown with a pair of line and arrow with affected band in FIG. 9A. Distribution of normalized rates of heterozygous SNVs in low-pass GS (FIG. 9B) and rates of heterozygous SNVs (FIG. 9C) in the same sample both show the decreased rates in the copy number deleted region. In FIGS. 9B and 9C, regions with consecutive decreased rates of heterozygous SNVs are indicated by red arrows with the numbers of windows involved in the bottom.

FIGS. 10A-10F show detection of AOH in chromosome 2 of sample HG00733. FIG. 10A shows distribution of copy-numbers among the windows (indicated by black dots) in chromosome 5 in this sample. The only deletion is shown by a purple arrow. X axis shows the genomic location in all figures, while Y axis indicates the copy-number in FIG. 10A. Distribution of normalized rates of heterozygous SNVs (FIG. 10B) and normalized rates of homozygous SNVs (FIG. 10C) cross chromosome 5 by low-pass GS. Distribution of rates of heterozygous SNVs (FIG. 10E) and rates of homozygous SNVs (FIG. 10F) cross chromosome 5 by GS. In FIGS. 10B and 10D, AOH (indicated by red arrows with the numbers of windows involved in the bottom) identified by observation of consecutive decreased rates of heterozygous SNVs. In FIGS. 10C and 10E, regions with consecutive increased rates of homozygous SNVs (indicated by blue arrows with the numbers of windows involved in the bottom). FIG. 10F shows distribution of parental genomic differences across chromosome 5. Y axis in FIGS. 10B-10F shows the rate of each corresponding parameter. Genomic region of the large AOH seq[GRCh37] 2p23.2p21 (29700000_42600000)x2 hmz is shown by a pair of green dotted lines across each figure.

FIGS. 11A-11D show AOH detected within a mosaic trisomy event in sample 16C0836 (FIG. 11A) Copy-number distribution reported by low-pass GS with windows indicated by black dots. The results confirmed approximately 40% increase of the whole chromosome 6 (indicated by a blue line). X axis in FIGS. 11A-11D indicates the genomic locations across chromosome 6, while in FIG. 11A, Y axis represents the copy-number. FIGS. 11B to 11D show the distribution of rates of “germline” heterozygous SNVs (AB), homozygous SNVs and “mosaic” heterozygous SNVs (AAB/ABB), respectively. In FIG. 11B, the candidate regions with AOH detected are indicated by each pair of red arrow and the number of windows, while in FIG. 11C, the windows with increased rate of homozygous SNVs within those regions reported in FIG. 11B are shown by each pair of blue arrow and the number of windows. In FIG. 11D, the candidate regions with increased rate of “mosaic” heterozygous SNVs are shown by each pair of a blue arrow and the number of windows.

FIGS. 12A-12E show AOH detected in sample aCGH15274. FIG. 12A shows copy-number distribution reported by low-pass GS with windows indicated by black dots. X axis in FIGS. 12A-12E indicates the genomic locations across chromosome 6, while in FIG. 12A, Y axis represents the copy-number. FIGS. 12B to 12D show the distribution of rates of “germline” heterozygous SNVs (AB), homozygous SNVs and “mosaic” heterozygous SNVs (AAB/ABB), respectively. In FIG. 12B, the candidate regions with AOH detected are indicated by each pair of red arrow and the number of windows, while in FIG. 12C, the windows with increased rate of homozygous SNVs within those regions reported in FIG. 12B are shown by each pair of blue arrow and the number of windows. In FIG. 12D, the candidate regions with increased rate of “mosaic” heterozygous SNVs are shown by each pair of a blue arrow and the number of windows. In FIG. 12E, Y axis shows maternally inherited genotype in the upper line (in black dots) and paternally inherited genotype in the bottom line (in black dots). The middle line shows in red if the rates of maternal/paternal genotypes are larger than 5 and in blue if the rates are smaller than 0.2.

FIGS. 13A-13D show rate distributions of different types of SNVs in deletion and duplication. FIG. 13A shows copy-number distribution reported by low-pass GS with windows indicated by black dots. CNV analysis result shows a deletion seq[GRCh37] del (8) (p23.3p23.2) chr8:g. 10134_5523520del and a duplication seq[GRCh37] dup (8) (q22.1q24.3) chr8:g.98620704_146298884dup in case 17BA0551. The X axis in FIGS. 13A-13D indicates the genomic locations across chromosome 8, while in (FIG. 13A), the Y axis represents the copy-number. FIGS. 13B to 13D show the distribution of rates of “germline” heterozygous SNVs (AB), homozygous SNVs and “mosaic” heterozygous SNVs (AAB/ABB), respectively. In FIG. 13B, the candidate regions with decreased rate of “germline” heterozygous SNVs are indicated by each pair of red arrow and the number of windows, while in FIG. 13C, the windows with increased rate of homozygous SNVs within those regions reported in FIG. 13B are shown by each pair of blue arrow and the number of windows. In FIG. 13D, the candidate regions with increased rate of “mosaic” heterozygous SNVs are shown by each pair of a blue arrow and the number of windows. The result shows that in 8p terminal deletion, all rates decreased, while in 8q terminal duplication, the rates of “mosaic” heterozygous SNVs increased.

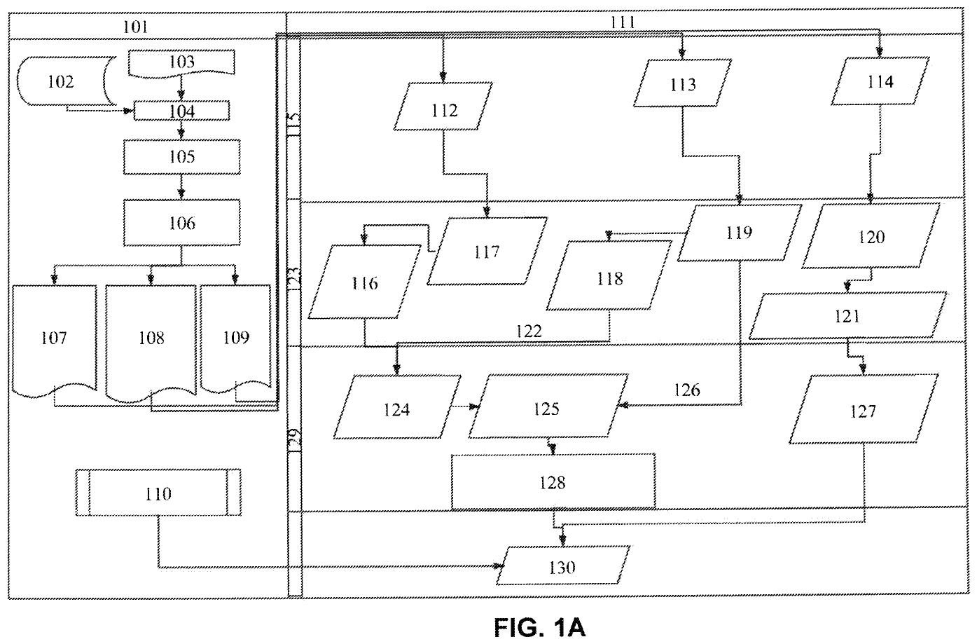

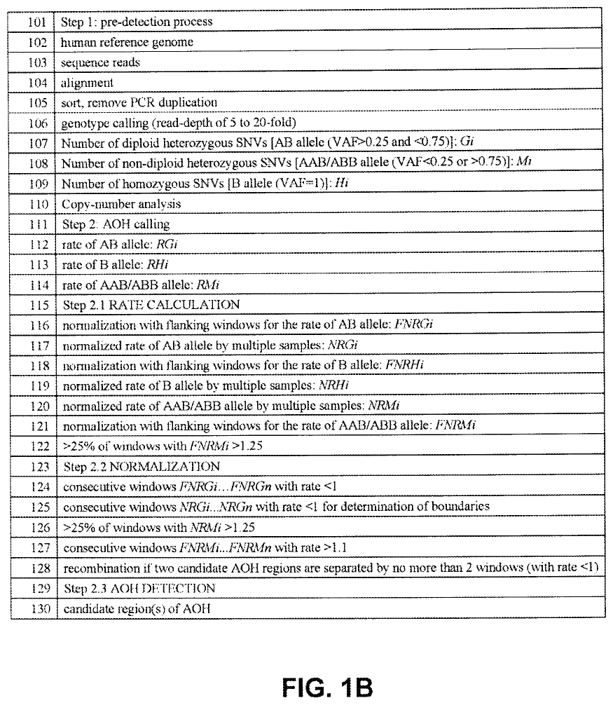

### DETAILED DESCRIPTION

Existing AOH detection methods usually require sequencing from either target-sequencing (e.g., exome sequencing) or genome sequencing (GS) (e.g., >30-fold). The target-sequencing method can be only applied to a particular region of the genome, while the GS method is costly for clinical practice.

AOH detection using a low-pass genome sequencing method has not been reported yet. Ideally, the principle of AOH detection is to identify those regions with consensus base type or expressed as homozygous base type. It will be commonly understood by a person skilled in the art that, for a low-pass genome sequencing method, it may be difficult to determine whether a site is truly both alleles mutated (homozygous) or the absence of reference allele is resulted from sequencing bias. Meanwhile, there will be “heterozygous SNVs” detected in those regions with AOH attributed to the high chance of false alignment. However, the rate of “heterozygous SNVs” would be decreased when there is a region with AOH. The inventors of the present application developed a method to apply low-pass GS to detect AOH utilizing the rate of heterozygous SNVs across genome or chromosome instead of identifying the absence of heterozygous base types or AB allele, and therefore completed the inventions described in the present application.

In a first aspect, there is provided in the present application a method of detecting absence of heterozygosity (AOH), e.g. copy-number neutral loss of heterozygosity (CN-LOH), in a biological sample from a subject, comprising
(i) receiving sequence reads from low-pass genome sequencing of genomic DNA of the biological sample;
(ii) aligning the sequence reads to a human genome reference, and selecting and sorting sequence reads aligned to the human genome reference based on the aligned chromosome and genomic coordinates;
(iii) identifying single-nucleotide variants (SNVs) in the aligned sequence reads, wherein a single-nucleotide variant at each site has a mutant base type different from the base type at the corresponding site from the human genome reference;
(iv) identifying homozygous SNVs, diploid heterozygous SNVs, or non-diploid heterozygous SNVs from the SNVs identified in step (iii), wherein
   a homozygous SNV is define based on the percentage of sequence reads supporting the mutant base type different from the base type at the corresponding site from the human genome reference being 100%,
   a diploid heterozygous SNV is define based on the percentage of sequence reads supporting the mutant base type different from the base type at the corresponding site from the human genome reference being no less than 25% and no large than 75%,
   a non-diploid heterozygous SNV is define based on the percentage of sequence reads supporting the mutant base type different from the base type at the corresponding site from the human genome reference being less than 25% and larger than 0% or larger than 75% and less than 100%;
(v) determining a rate of homozygous SNVs, diploid heterozygous SNVs, or non-diploid heterozygous SNVs identified in step (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) comparing the rate of homozygous SNVs, diploid heterozygous SNVs, or non-diploid heterozygous SNVs for individual windows determined from step (v) with an average rate of homozygous SNVs, diploid heterozygous SNVs, or non-diploid heterozygous SNVs for corresponding individual windows established from a control population.

In some embodiments, the biological sample is selected from the group consisting of peripheral blood, chorionic villus, amniotic fluid, cord blood, placental tissue, and tissue samples from organs. In some embodiments, the subject is a pregnant female, an infant, a subject suffering from a cancer, or a subject suspected of suffering from a cancer. As understood by a person skilled in the art, detection of AOH is useful in various settings, e.g. prenatal genetic diagnosis, postnatal genetic diagnosis, or even cancer genetics. Therefore, subject candidates or suitable biological samples can be determined by a person skilled in the art depending on the purpose for AOH detection.

Either single-end sequence reads or paired-end sequence reads (also referred to as “read-pairs”) are well known to a person skilled in the art, and can be suitably used in the present application.

As compared with GS requiring sequencing, the low-pass genome sequencing in the present application may have a lower read depth, e.g. 3˜5 folds, such as 3 folds. Suitable human genome reference for alignment step can be selected by a person skilled in the art. In a particular embodiment, the human genome reference is hg19/GRCh37 or hg38/GRCh38.

Suitable human genome reference for alignment step can also be selected by a person skilled in the art, including, but not limited to, Short Oligonucleotide Alignment Program 2 (SOAP2) or Burrows-Wheeler Aligner (BWA) and Bowtie2. Default setting can be adopted.

In some embodiments, step (ii) further includes removing sequence reads due to polymerase chain reaction (PCR) duplication.

In some embodiments, step (iii) further includes discarding a site as described below:
(a) a minimal read-depth of the site is determined by the minimal read-depth of the biological sample;
(b) a maximum read-depth of the site is determined by the maximal read-depth of the biological sample; or
(c) a site where no sequence read supports a mutant base type.

In some embodiments, the window in step (v) has a fixed length, e.g. 100 kb.

In some embodiments, step (v) comprises
determining the number of homozygous SNVs, diploid heterozygous SNVs, or non-diploid heterozygous SNVs for the window,
determining the average number of homozygous SNVs, diploid heterozygous SNVs, or non-diploid heterozygous SNVs among all windows in the biological sample, and
calculating the rate of homozygous SNVs, diploid heterozygous SNVs, or non-diploid heterozygous SNVs for the window by dividing the number of homozygous SNVs, diploid heterozygous SNVs, or non-diploid heterozygous SNVs identified for the window by the average number of homozygous SNVs, diploid heterozygous SNVs, or non-diploid heterozygous SNVs among all windows in the biological sample.

In some embodiments, the control population has the same gender as the subject. In some embodiments, the control population has at least 30 control subjects.

Theoretically, AOH is defined as absence of heterozygosity or runs of homozygosity presented in diploid chromosomes when copy-number is neutral (no deletion encountered). For a male subject, only autosomal chromosomes are diploid, while for a female subject, both autosomal chromosomes and sex chromosomes are diploid. Therefore, the control population can include control subjects with the same gender as the test subject.

In some embodiments, step (vi) comprises normalizing the rate of homozygous SNVs, diploid heterozygous SNVs, or non-diploid heterozygous SNVs for a window by an average rate of homozygous SNVs, diploid heterozygous SNVs, or non-diploid heterozygous SNVs for the corresponding window established from the control population, thereby providing a corresponding rate ratio of homozygous SNVs, diploid heterozygous SNVs, or non-diploid heterozygous SNVs for the window.

In some embodiments, in step (vi), increased rate of non-diploid heterozygous SNVs indicates mosaic AOH, and preferably, step (vi) further comprises where the copy-number mosaic duplication represented as copy-ratio larger than 1 or the copy-number neutral expressing as copy-ratios equal to 1, for all windows with non-diploid heterozygous SNVs rate ratios larger than 1, defining a region if there are a plurality of windows with consecutive non-diploid heterozygous SNVs rate ratios larger than 1.15; and reporting the region as presence of mosaic AOH.

In some embodiments, in step (vi), decreased rate of diploid heterozygous SNVs and increased rate of homozygous SNVs indicate AOH, and preferably, step (vi) further comprises
where the copy-number neutral expressing as copy-ratios equal to 1, for all windows with diploid heterozygous SNVs rate ratios less than 1, defining a region if there are a plurality of windows with consecutive diploid heterozygous SNVs rate ratios less than 0.5 and the percentage of windows with homozygous rate ratios larger than 1.25 is at least 25%, and optionally combining two regions into one if there are no more than one windows with diploid heterozygous SNVs rate ratios larger than 0.5 but less than 1; and
reporting the region as presence of AOH.

In some embodiments, an average rate of heterozygous SNVs for corresponding individual windows established from a control population is determined by (ci) receiving sequence reads from low-pass genome sequencing of genomic DNA of a biological sample from a control subject from the control population:
(cii) aligning the sequence reads to a human genome reference, and selecting and sorting sequence reads aligned to the human genome reference based on the aligned chromosome and genomic coordinates;
(ciii) identifying single-nucleotide variants (SNVs) in the aligned sequence reads, wherein a single-nucleotide variant at each site has a mutant base type different from the base type at the corresponding site from the human genome reference;
(civ) identifying homozygous SNVs, diploid heterozygous SNVs, or non-diploid heterozygous SNVs from the SNVs identified in step (ciii), wherein
   a homozygous SNV is define based on the percentage of sequence reads supporting the mutant base type different from the base type at the corresponding site from the human genome reference being 100%,
   a diploid heterozygous SNV is define based on the percentage of sequence reads supporting the mutant base type different from the base type at the corresponding site from the human genome reference being no less than 25% and no large than 75%,
   a non-diploid heterozygous SNV is define based on the percentage of sequence reads supporting the mutant base type different from the base type at the corresponding site from the human genome reference being less than 25% and larger than 0% or larger than 75% and less than 100%;
(cv) determining a rate of homozygous SNVs, diploid heterozygous SNVs, or non-diploid heterozygous SNVs identified in step (civ) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(cvi) averaging rates of homozygous SNVs, diploid heterozygous SNVs, or non-diploid heterozygous SNVs for a window from all control subjects to provide an average rate of homozygous SNVs, diploid heterozygous SNVs, or non-diploid heterozygous SNVs for the corresponding window in the control population.

In some embodiments, the method further comprises, between step (cii) and (ciii), a step of sex determination, wherein the aligned ratios of chromosome X, chromosome Y and the whole genome are calculated as the numbers of sequence reads aligned to the chromosome/genome dividing by the length defined by the humane reference genome, respectively, the chromosome Y percentage is calculated as the aligned ratio of chromosome Y dividing by the aligned ratio of the whole genome, and a control subject is considered as male if the chromosome Y percentage is larger than 0.05.

In some embodiments, steps (ciii) to (cvi) are carried out on male and female control subjects respectively, based on the result of the step of sex determination.

In some embodiments, in step (cvi), if rates of homozygous SNVs, diploid heterozygous SNVs, or non-diploid heterozygous SNVs for a window among control subjects have substantial deviation, the average rate of homozygous SNVs, diploid heterozygous SNVs, or non-diploid heterozygous SNVs for the window is calculated as an average of the rates of the window and its flanking windows (e.g., two upstream and two downstream windows).

As a non-limiting example, a process from establishing building up a control dataset to detection of AOH in a case sample is described below.

### Building Up a Control Dataset

### (i) Alignment

For each sample, single-end reads or paired-end reads are subjected for alignment to the human genome reference (such as GRCh37/hg19 or GRCh38/hg38) by the alignment softwares [i.e., Short Oligonucleotide Alignment Program 2 (SOAP2), Burrows-Wheeler Aligner (BWA) and Bowtie2] with default setting. All the reads/read-pairs aligned to the human genome reference are selected, and sorted based on the aligned chromosome and coordinates, followed by removal of reads/read-pairs due to polymerase chain reaction (PCR) duplication. The remained reads/read-pairs are named as processed reads/read-pairs are subjected for further analysis.

### (ii) Sex Determination

The aligned ratios of chromosome X, chromosome Y and the whole genome are calculated as the numbers of reads/read-pairs aligned to the certain chromosome/genome dividing by the length (defined by the humane reference genome), respectively. The chromosome Y percentage is calculated as the aligned ratio of chromosome Y dividing by the aligned ratio of the whole genome, and a case would be considered as male if the chromosome Y percentage larger than 0.05. After sex determination, a minimal of 30 cases from each sex are selected for control construction independently.

(iii) Putative Single-Nucleotide Variants (SNVs) Calling

The processed reads/read-pairs from step (i) are used as input for identifying the alignment result in each coordiante by MPileup module from Samtools. From each site, the aligned information may presents as:
a. “.” is with consistent base type as human genome reference and the aligned strand is plus or “+”;
b. “,” is with consistent base type as human genome reference and the aligned strand is minus or
c. “A” (using base type “A” as example) is with mutant base type different from the base type from human genome reference and the aligned strand is plus or “+”;
d. “a” (using base type “A” as example) is with mutant base type different from the base type from human genome reference and the aligned strand is minus or “−”.

From each site, the chromosome, coordinate, base type in reference and the aligned information are subjected for putative SNVs detection and the following sites can be discarded:
a. A minimal read-depth of each “putative” site is determined by the minimal read-depth of the particular sample. For example, when there is only 3-fold for a case, those sites with read-depth <3 can be discarded. In addition, given the sequencing read-depth is following a normal distribution, those sites with extremely higher read-depth such as >mean+3SD (standard deviations) can be also discarded since they are mostly likely resulted from systematic errors; or
b. No read supporting a mutant base type;
(iv) Rate of homozygous or “germline”/“mosaic” heterozygous SNVs

Genome-wide fixed window (such as 100-kb) can be used. For a window Wᵢ, the number of homozygous or “germline”/“mosaic” heterozygous SNVs Hi/Gi/Mi identified in step (iii) can be counted, while the average of the corresponding type of SNVs among all the windows in a certain sample would be counted as RH/RG/RM. For Wᵢ, the rate of homozygous or “germline”/“mosaic” heterozygous SNVs RHi/RGi/RMi can be calculated as Hi/Gi/Mi dividing by RH/RG/RM.

For building up the control dataset, for each sex, the average rate of a window Wᵢ among all the control samples can be calculated as the average value of RHi/RGi/RMi named NRHi/NRGi/NRMi. The average rate of each window among all the whole genome can be kept for future population based normalization of a case sample.

### Detection of AOH in Case Sample

### (i) Data Preparation

For a case C, the reads/read-pairs undergo alignment, sorting, removal of PCR duplication, sex determination, putative SNVs calling and rate of homozygous or “germline”/“mosaic” heterozygous SNVs determination.

Afterwards, for each window Wᵢ, the rate of homozygous or “germline”/“mosaic” heterozygous SNVs NRHi/NRGi/NRMi is normalized by the average value of this windown NRHia/NRGia/NRMia from the corresponding sex control cohort named NRHic/NRGic/NRMic. Given a high deviation of NRHic/NRGic/NRMic, the average value of four flanking windows (two upstream and two downsteam) with Wᵢ itself NRHic/NRGic/NRMic are assigned to be the normalized rate in Wᵢ.

(ii) Screening of the Candidate Region with AOH.

Putative AOH can be defined as the region/window with NRGic less than 0.5, and the windows with NRGic less than 0.5 are selected.

(iii) Breakpoint Determination

For all windows with NRGic less than 0.5, a region is defined if there are a number of windows with consecutive NRGic less than 0.5, while the percentage of windows with NRHic larger than 1.25 should be more than 25%. In addition, two regions can be combined if there are only less than one window with NRGic larger than 0.5 but less than 1. The final region(s) with AOH can be reported after window/region combination. The resolution of this detection may be as small as 2.5 Mb.

In a second aspect, there is provided in the present application a computer system for detecting absence of heterozygosity (AOH), e.g. copy-number neutral loss of heterozygosity (CN-LOH), in a biological sample from a subject, comprising a processor and a memory storing a plurality of instructions, wherein the processor, upon processing the instructions, is configured to:
(i) receive sequence reads from low-pass genome sequencing of genomic DNA of the biological sample;
(ii) align the sequence reads to a human genome reference, and selecting and sorting sequence reads aligned to the human genome reference based on the aligned chromosome and genomic coordinates;
(iii) identify single-nucleotide variants (SNVs) in the aligned sequence reads, wherein a single-nucleotide variant at each site has a mutant base type different from the base type at the corresponding site from the human genome reference;
(iv) identify homozygous SNVs, diploid heterozygous SNVs, or non-diploid heterozygous SNVs from the SNVs identified in (iii), wherein
   a homozygous SNV is define based on the percentage of sequence reads supporting the mutant base type different from the base type at the corresponding site from the human genome reference being 100%,
   a diploid heterozygous SNV is define based on the percentage of sequence reads supporting the mutant base type different from the base type at the corresponding site from the human genome reference being no less than 25% and no large than 75%,
   a non-diploid heterozygous SNV is define based on the percentage of sequence reads supporting the mutant base type different from the base type at the corresponding site from the human genome reference being less than 25% and larger than 0% or larger than 75% and less than 100%;
(v) determine a rate of homozygous SNVs, diploid heterozygous SNVs, or non-diploid heterozygous SNVs identified in (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) compare the rate of homozygous SNVs, diploid heterozygous SNVs, or non-diploid heterozygous SNVs for individual windows determined from (v) with an average rate of homozygous SNVs, diploid heterozygous SNVs, or non-diploid heterozygous SNVs for corresponding individual windows established from a control population.

In a third aspect, there is provided in the present application a computer readable medium storing a plurality of instructions, wherein the plurality of instructions, upon executed by one or more processors, perform an operation including
(i) receiving sequence reads from low-pass genome sequencing of genomic DNA of a biological sample from a subject;
(ii) aligning the sequence reads to a human genome reference, and selecting and sorting sequence reads aligned to the human genome reference based on the aligned chromosome and genomic coordinates;
(iii) identifying single-nucleotide variants (SNVs) in the aligned sequence reads, wherein a single-nucleotide variant at each site has a mutant base type different from the base type at the corresponding site from the human genome reference;
(iv) identifying homozygous SNVs, diploid heterozygous SNVs, or non-diploid heterozygous SNVs from the SNVs identified in (iii), wherein
   a homozygous SNV is define based on the percentage of sequence reads supporting the mutant base type different from the base type at the corresponding site from the human genome reference being 100%,
   a diploid heterozygous SNV is define based on the percentage of sequence reads supporting the mutant base type different from the base type at the corresponding site from the human genome reference being no less than 25% and no large than 75%,
   a non-diploid heterozygous SNV is define based on the percentage of sequence reads supporting the mutant base type different from the base type at the corresponding site from the human genome reference being less than 25% and larger than 0% or larger than 75% and smaller than 100%;
(v) determining a rate of homozygous SNVs, diploid heterozygous SNVs, or non-diploid heterozygous SNVs identified in (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) comparing the rate of homozygous SNVs, diploid heterozygous SNVs, or non-diploid heterozygous SNVs for individual windows determined from (v) with an average rate of homozygous SNVs, diploid heterozygous SNVs, or non-diploid heterozygous SNVs for corresponding individual windows established from a control population.

In a fourth aspect, there is provided in the present application a device comprising one or more processors and a computer readable medium of the third aspect.

The features or embodiments described in the first aspect can be applied to or combined into the second to fourth aspects.

It should be understood that any of the embodiments of the present invention can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Additionally, details of any specific embodiment may not always be present in variations of that embodiment or may be added to other embodiments.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

A recitation of “a”, “an” or “the” is intended to mean “one or more” unless specifically indicated to the contrary. The use of “or” is intended to mean an “inclusive or,” and not an “exclusive or” unless specifically indicated to the contrary.

All patents, patent applications, publications, and descriptions mentioned herein are incorporated by reference in their entirety for all purposes. None is admitted to be prior art.

### Example

### Methods

### Subject Enrollment and Sample Recruitment

GS data [paired-end 126-bp, from Illumina platform (San Diego, CA, United States), >30-fold, hereafter referred as GS] of three trios (proband-father-mother) from the 1000 Genomes Project and 50 cases with increased nuchal translucency sequenced [paired-end 100-bp, from MGISEQ-2000 (MGI, BGI-Shenzhen, Shenzhen, China)] in our previous study [27] were used for the method development and validation. In addition. 12 DNA samples from 10 cases with AOH reported by CMA were also recruited for low-pass GS (˜4-fold). Written informed consent was obtained from each participant (Table 1). Parental DNA samples were also obtained for two cases (Table 1).

TABLE 1
Detection Results of AOH by Chromosomal Microarray Analysis and Low-pass GS

| Sample | Sample | | | |
|---|---|---|---|---|
| ID | source | Indications | CMA results | Low-pass GS results |
| 15C0187 | AF | NIPT: | arr[GRCh37] | Batch 1: seq[CRCh37] |
| | | increased | 2p25.3p25.1(15703_9969475) × | 2p25.3p25.1(100000_8800000) × |
| | | amount of | 2 hmz; | 2 hmz; |
| | | chromosome | 2p14p11.2(65459100_89126216) × | 2p14p11.2(66600000_89000000) × |
| | | 2 | 2 hmz; | 2 hmz; |
| | | | 2q11.1q14.1(95665733_114718893) × | |
| | | | 2 hmz; | 2q11.1q14.1(98500000_113600000) × |
| | | | 2q31.1q33.3(176973082_207693815) × | 2 hmz; |
| | | | 2 hmz | 2q31.1q33.3(177100000_206800000) × |
| | | | | 2 hmz |
| | | | | Batch 2 seq[CRCh37] |
| | | | | 2p25.3p25.1(100000_9900000) × |
| | | | | 2 hmz; |
| | | | | 2p14p11.2(66600000_89700000) × |
| | | | | 2 hmz; |
| | | | | 2q11.1q14.1(96800000_113400000) × |
| | | | | 2 hmz; |
| | | | | 2q31.1q33.3(176900000_206800000) × |
| | | | | 2 hmz |
| 15C0774 | AF | Bilateral | multiple chromosomes | multiple chromosomes |
| | | club foot; | showed LOH (266-Mb) | showed LOH (273.4-Mb) |
| | | previous | | |
| | | child/ | | |
| | | pregnancy | | |
| | | with fetal | | |
| | | anomalies | | |
| 16C0067 | AF | NIPT: | arr[GRCh37] | seq[GRCh37] |
| | | increased | 15q21.3q26.1(57667310_89281864) × | 15q21.3q22.31(54400000_63900000) × |
| | | chromosome | 2 hmz | 2 hmz; |
| | | 15 | | 15q22.31q25.3(66100000_88100000) × |
| | | | | 2 hmz |
| 16C0836 | Tissues | Advanced | arr[GRCh37] | Batch 1: seq[GRCh37] |
| | | maternal | 7p22.3q36.3(109626_158928217) × | 7p22.3q36.3(400000_159100000) × |
| | | age | 2~3 hmz | 2~3 hmz |
| | | | | Batch 2: seq[GRCh37] |
| | | | | 7p22.3q36.3(300000_157900000) × |
| | | | | 2~3 hmz |
| 17C0705 | Tissues | Fetal | multiple chromosomes | Batch 1: multiple chromosomes |
| | | anomalies: | showed LOH (270.5-Mb) | showed LOH (286.4-Mb) |
| | | anencephaly? | | Batch 2: multiple chromosomes |
| | | abnormal | | showed LOH (287.4-Mb) |
| | | heart | | |
| | | (Right | | |
| | | heart more | | |
| | | prominent) | | |
| | | receding | | |
| | | chin | | |
| | | angulation | | |
| | | over | | |
| | | lumbar spine. | | |
| | | Both | | |
| | | hands are | | |
| | | claw legs | | |
| | | persistently | | |
| | | adducted. | | |
| 17C1122 | CVS | Couples with | multiple chromosomes | Batch 1: multiple chromosomes |
| | | previous | showed LOH (139-Mb) | showed LOH (145.7-Mb) |
| | | child with | | Batch 2: multiple chromosomes |
| | | Progressive | | showed LOH (122.4-Mb) |
| | | Myoclonic | | |
| | | Epilepsy- | | |
| | | 3 with or | | |
| | | without | | |
| | | intracellular | | |
| | | inclusion | | |
| 17C1175 | blood | Affected | multiple chromosomes | multiple chromosomes showed LOH |
| | | sibling of | showed LOH (132-Mb) | (161.7-Mb) |
| | | 17C1122 | | |
| 17C1176 | blood | Unaffected | multiple chromosomes | Batch 1: multiple chromosomes |
| | | sibling of | showed LOH (183-Mb) | showed LOH (168.5-Mb) |
| | | 17C1122 | | Batch 2: multiple chromosomes |
| | | | | showed LOH (171.8-Mb) |
| 18C1841 | AF | Down | arr[GRCh37] | seq[GRCh37] |
| | | Syndrome | 6p12.1(55098894_58614061) × | dup(6)(p12.1p11.1)chr6: g.55090143 |
| | | Screening: | 3; | 58771838dup; |
| | | high risk; | 6p25.2q27(3568005_170712179) × | 6p25.2q27(300000_170300000) × |
| | | NIPT: | 2 hmz dn | 2 hmz dn |
| | | increase | | |
| | | risk of | | |
| | | trisomy 6 | | |
| 18C1493 | CVS | Fetal | arr[GRCh37] | seq[GRCh37] |
| | | anomalies: | 6p25.3q27(400187_170890108) × | 6p25.3q27(200000_170600000) × |
| | | right | 2~3 hmz | 2~3 hmz |
| | | Multicystic | | |
| | | dysplastic | | |
| | | kidney | | |
| 18C1564 | AF | AF of | arr[GRCh37] | seq[GRCh37] |
| | | 18C1493 | 6p25.3p22.3(211941_24108657) × | 6p25.3p22.3(1200000_23600000) × |
| | | | 2 hmz; | 2 hmz; |
| | | | 6p21.1p11.2(41959702_58437809) × | 6p21.1p11.2(42400000_58800000) × |
| | | | 2 hmz; | 2 hmz; |
| | | | 6q11.1q15(62009546_89885237) × | 6q11.1q15(62100000_89900000) × |
| | | | 2 hmz; | 2 hmz; |
| | | | 6q22.3q25.1(128356873_151322367) × | 6q22.3q25.1(127100000_152600000) × |
| | | | 2 hmz | 2 hmz |
| aCGH15274 | cord | Fetal | arr[GRCh37] | seq[GRCh37] |
| | blood | cord | 6p25.3p22.3(206528_23592348) × | 6p25.3p22.3(500000_23600000) × |
| | | blood of | 2 hmz, | 2 hmz; |
| | | 18C1493 | 6p21.1q15(42131875_89170082) × | 6p21.1p11.2(42300000_58800000) × |
| | | | 2 hmz, | 2 hmz; |
| | | | 6q23.1q25.2(130747461 152637678) × | 6q11.1q15(62400000_89900000) × |
| | | | 2 hmz | 2 hmz; |
| | | | | 6q22.3q25.1(127100000_152600000) × |
| | | | | 2 hmz |

### DNA Preparation and Routine CMA

Genomic DNA from chorionic villi, amniotic fluids or fetal cord blood was extracted using the DNeasy Blood & Tissue Kit (Cat No./ID: 69506, Qiagen, Hilden, Germany) at the time of CMA testing. DNA was quantified with the Qubit dsDNA HS Assay kit (Invitrogen, Carlsbad, CA), and the DNA integrity was assessed by agarose gel electrophoresis.

For routine CMA testing, we employed a well-established customized CMA 8×60k Fetal DNA Chip v2.0 (Agilent Technologies, Santa Clara, CA, United States), containing both SNP and comparative genomic hybridization (CGH) probes [28, 29]. The experiments were performed according to the manufacturer′ protocol. CNV and AOH analyses were performed with the CytoGenomics software [28, 29].

### Low-Pass GS

100-ng of genomic DNA from each sample was sheared to a fragment size ranging from 300˜500-bp with the Covaris S2 Focused Ultrasonicator (Covaris, Inc., Woburn, MA, United States). Library construction protocol included end-repairing, A-tailing, adapter-ligation and PCR amplification. The PCR products were subsequently heat-denatured to form single strand DNA, followed by circularization with DNA ligase. After construction of the DNA nanoballs, paired-end sequencing with 100-bp at each end was carried out with a read-depth of ˜4-fold for each sample on the MGISEQ-2000 platform (MGI) [30]. For evaluation of reproducibility, low-pass GS including library construction and sequencing was replicated for five samples (Table 1).

### Data Analysis and Detection of SNVs

QC for the paired-end reads was assessed via FastQC (https://www.bioinformatics.babraham.ac.uk/projects/fastqc/) and subsequently aligned to the human reference genome (GRCh37/hg19) by Burrows-Wheeler Aligner (BWA) [31]. The alignment file was reformatted, and the reads suspected to be resulted from PCR duplication were removed both by SAMtools [32]. For GS, SNV detection was performed with HaplotypeCaller v3.4 from the Genome Analysis Toolkit (GATK, Broad Institute) and classification of homozygous and heterozygous SNVs was conducted by ANNOVAR [34]. Of note that since the SNVs detected by GATK HaplotypeCaller module was based on the diploid setting, all heterozygous SNVs reported by GS were classified as “germline” heterozygous SNVs for further analysis.

For each set of GS (>30-fold), low-pass GS (4-fold) was simulated by generating by random selection of paired-end reads [24]. For low-pass GS data either by in silico simulation or sequencing, paired-end reads were subjected to the alignment, reformatting, removal of PCR duplication following the methods mentioned above. Afterwards, the coverage of mapped reads with genotypic information at each genomic location was summarized by the mpileup module from SAMtools [32], and the sites with reads supporting a mutant base type were selected and defined as SNVs. SNVs were classified into three categories based on the variant allele fraction (VAF), which was calculated as the number of reads supporting the mutant base type dividing by the total number of reads supporting in this particular locus: (1) a homozygous SNV was defined if no reads support the wild-type allele (the percentage of sequence reads supporting the mutant base type is 100%): (2) a “germline” heterozygous SNVs was classified if its VAF was no less than 25% and no more than 75%; and (3) a “mosaic” heterozygous SNV was detected if its VAF was smaller than 25% and larger than 0% or larger than 75% and smaller than 100%.

### Calculation of Parental Genomic Difference

For the three trios with GS data downloaded from the 1000 Genomes Project, the genotypic information from each parental sample was also obtained from GATK. The number of SNVs in which both parents are homozygous for different genotypes were counted as P_{d} with fixed windows (100-kb in size), while the total number of SNVs detected was also counted as Pₜ in the same windows. Rate of parental genomic difference in each window was calculated as P_{d} dividing by Pₜ as P_{dr}.

### Rates of Different Types of SNVs

For each kind of SNV detected by either GS or low-pass GS, the population-based normalized rate of homozygous SNVs with a fixed window size of 100-kb was calculated as: (1) for a particular window Wᵢ, the number of homozygous SNVs Hᵢ was counted based on the genomic locations: (2) Hᵢ was then normalized by the average number of homozygous SNVs among all windows in this case set as RHᵢ; and (3) further normalized by the average rate of homozygous SNVs among all cases in this particular window and set as NRHᵢ. The population-based normalized rate of “germline” heterozygous SNVs (NRGᵢ) and “mosaic” heterozygous SNVs (NRMᵢ) were calculated in the same way as NRHᵢ, respectively.

### CNV and AOH Detection

CNV detection was conducted based on our previous studies [22, 35]. Since the in-house reference cohort was developed using data generated from single-end reads with 50-bp, only read 1 (or named 1^{st} end) of each pair was used and trimmed to 50-bp for CNV analysis. In brief, adjustable sliding windows (50-kb with 5-kb increment) were used to report the candidate region(s) for CNV(s), and adjustable non-overlapping windows (5-kb) were used for identifying the precise boundaries by the method of increment-ratio-of-coverage. Rare CNVs were reported if the P value of population-based U-test less than 0.0001.

For detecting a AOH with GS, a region of AOH was reported if consecutive windows were with NRGᵢ less than 0.4 and 50% of these windows were with NRHᵢ larger than 1.25. In addition, two candidate regions (larger than 200-kb) were combined if they were separated by one window, whose NRGᵢ was larger than 0.4 but less than 1. A final region with AOH >500-kb was reported based on the recommendation from the International System for Human Cytogenomic Nomenclature (ISCN, 2016).

Detection of AOH with low-pass GS was performed by setting NRGᵢ as the average value of four flanking regions (two upstream and two downstream) and itself to give FNRGᵢ, while each NRHᵢ was also set as the average value of eight flanking regions (two upstream and two downstream) and itself as FNRHᵢ. A candidate region with AOH was reported if consecutive windows were with FNRGᵢ less than 0.5 and also FNRHᵢ values were larger than 1.25 for 25% of the windows within the candidate region. Further, determination of precise boundaries was performed when there is a region with consecutive NRGᵢ values less than 0.5 and 25% of windows with NRHᵢ larger than 1.25 inside a candidate region. In addition, two candidate regions (larger than 200-kb) were combined if they were separated by one window, whose NRGᵢ value was larger than 0.5 but less than 1. A final region with AOH >500-kb was reported also following ISCN 2016.

For detecting AOH within a mosaic trisomy event by low-pass GS, each NRMi was further set as the average value of four flanking regions (two upstream and two downstream) and itself to give FNRMᵢ. A region with consecutive NRMi larger than 1.15 was reported when the size was >1-Mb.

### Determination of Parental Origin

For the two cases with parental low-pass GS available, SNVs detection was performed for each parent in each family with the method same as in proband. Only the loci where the parents were homozygous for different genotypes were selected. The number of maternal/paternal origin SNVs, which was defined as the proband having at least one allele consistent with the mother/father. The ratio of maternal origin SNVs divided by paternal origin was calculated in each fixed window with 1-Mb in size and the regions with extreme value (rate >5 or <0.2) was reported.

### Quantitative Fluorescent-PCR

The parental origins of chromosomes 6 and 15 reported by low-pass GS were further validated by Quantitative Fluorescent-PCR (QF-PCR) with short tandem repeat (STR) markers selected from UCSC genome browser following the manufacturer's instructions as described in our previous study [36].

### AOH Validation

For those three trios GS data downloaded from the 1000 Genomes Project, raw data (idat files) from SNP array platform Omni 2.5 M (Illumina) were downloaded from ftp://ftp.1000genomes.ebi.ac.uk/vol1/ftp/release/20130502/supporting/hd_genotype_c hip/broad_intensities/ [37] and imported for AOH detection by GenomeStudio (Illumina) with defaulted setting for the detection parameters and resolution (1-Mb) [5].

### Results

In this study, we first evaluated the sensitivity and specificity of AOH detection with low-pass GS by using GS as reference and further validated this method with 12 clinical samples with known AOH results reported by CMA.

### Detection of AOH by the Rates of Heterozygous/Homozygous SNVs

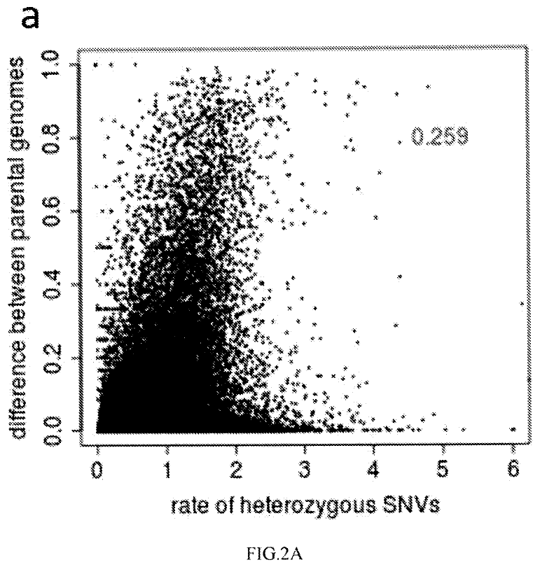

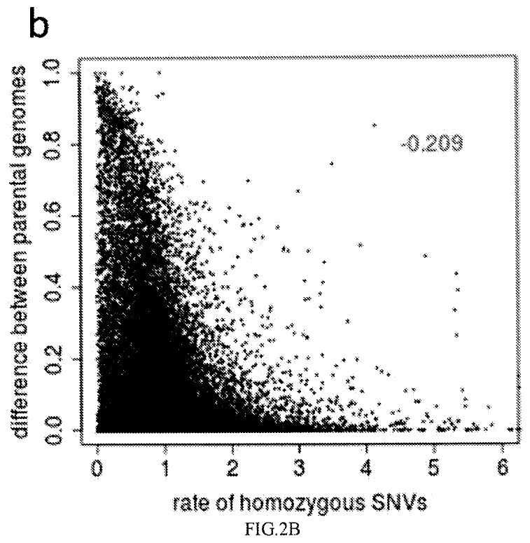

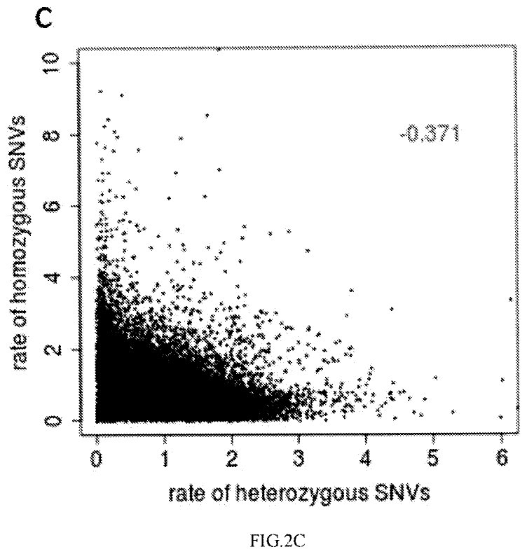

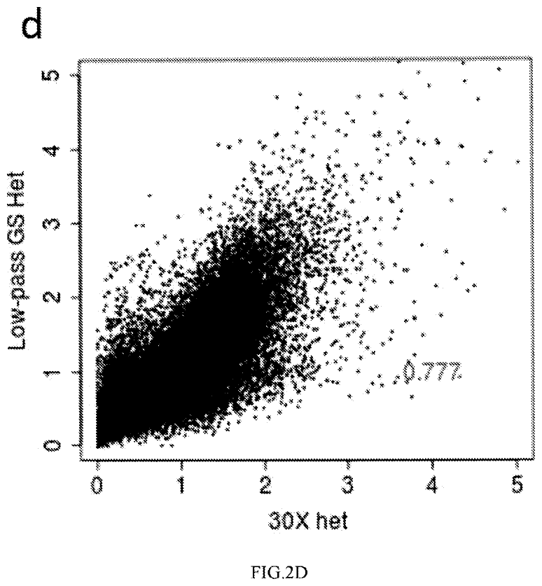

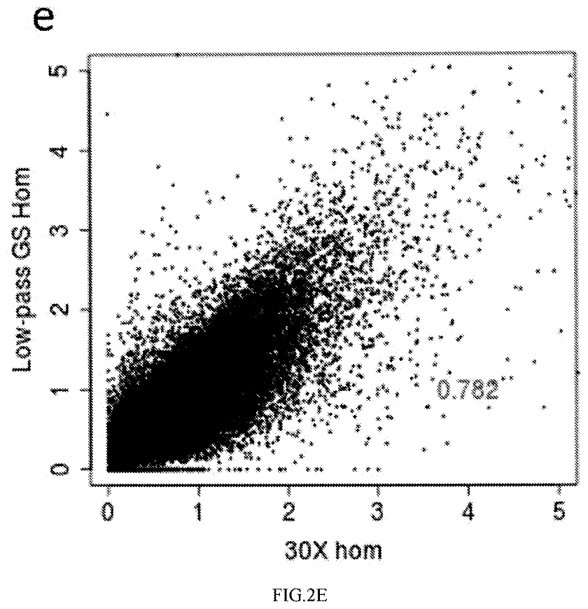

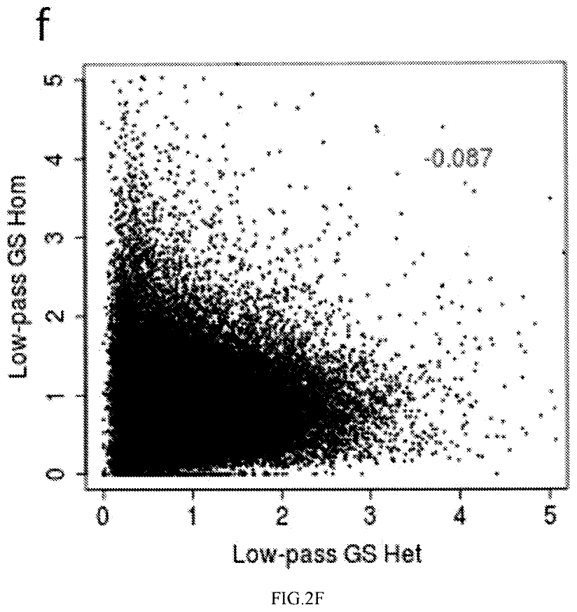

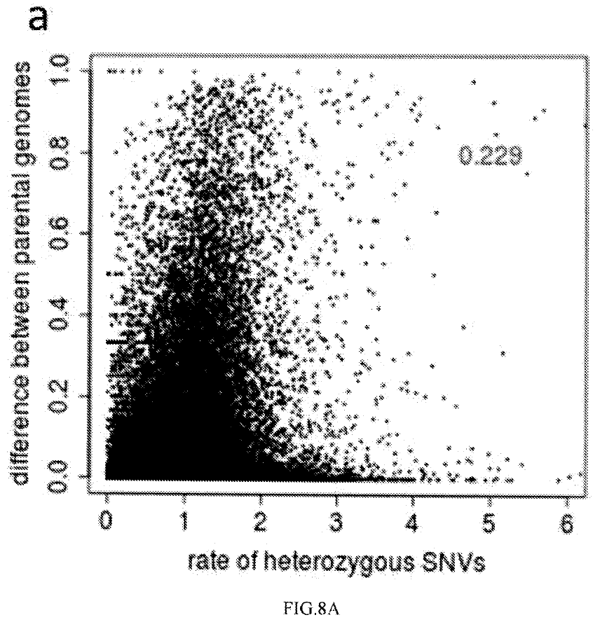

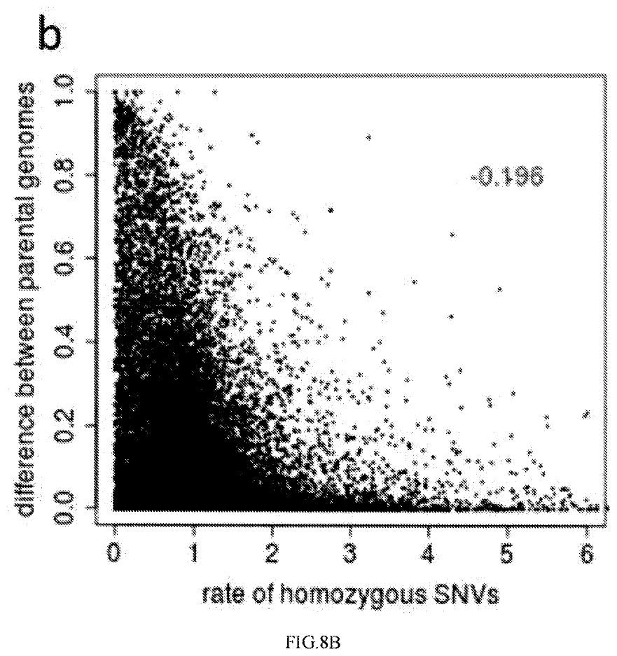

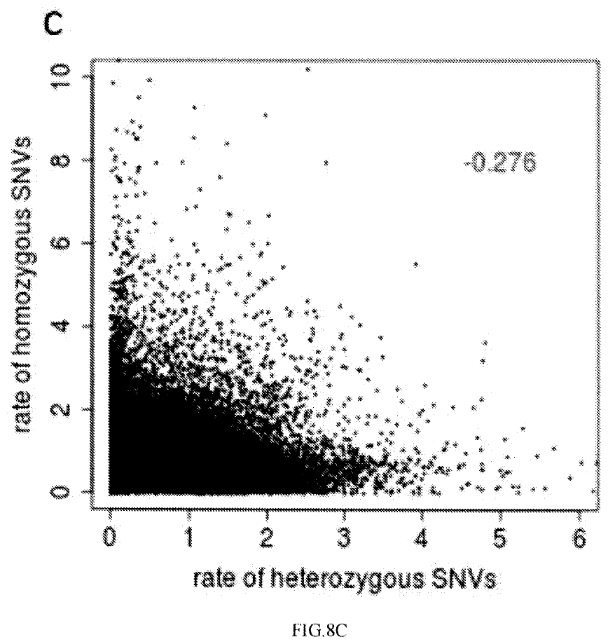

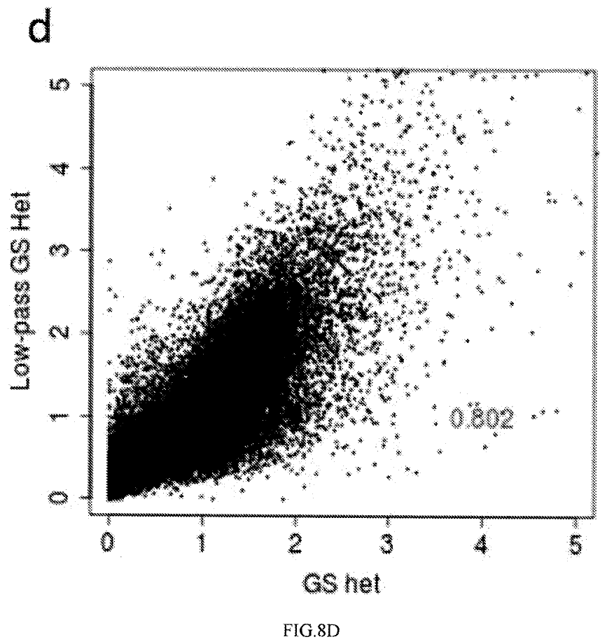

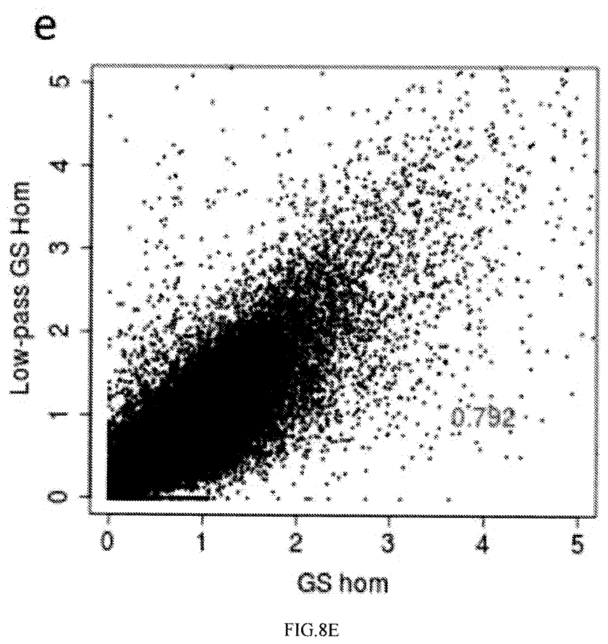

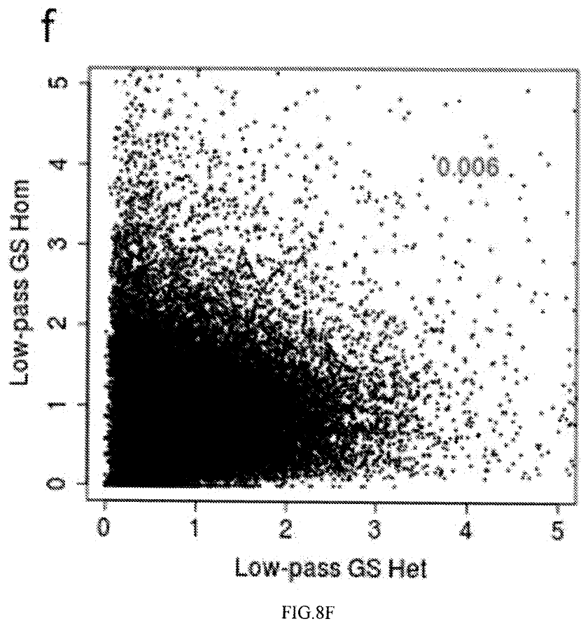

As AOH represents runs of homozygosity commonly resulting from identity-by-descent, we evaluated whether the similarity of parental genome could be indicated by the rates of heterozygous and homozygous SNVs detected. Since it would be difficult to determine the similarity of parental genotypes, parental genomic differences were used instead. The results from GS indicated that the rates of parental genomic differences were positively correlated with the rates of heterozygous SNVs (FIG. 2A and FIG. 8A) and negatively correlated with the rates of homozygous SNVs (FIG. 2B and FIG. 8B). It indicated that decreasing parental genomic differences would result in decreased rates of heterozygous SNVs and increased rates of homozygous SNVs, while the rates of heterozygous and homozygous SNVs were negatively correlated (FIG. 2C and FIG. 8C).

In addition, the results also showed that both rates of homozygous/heterozygous SNVs from GS were strongly correlated with the ones from low-pass GS (FIGS. 2D-2F and FIGS. 8D-8F), demonstrating the feasibility of detecting AOH using low-pass GS.

### Evaluation of Sensitivity and Specificity in Detection of AOH

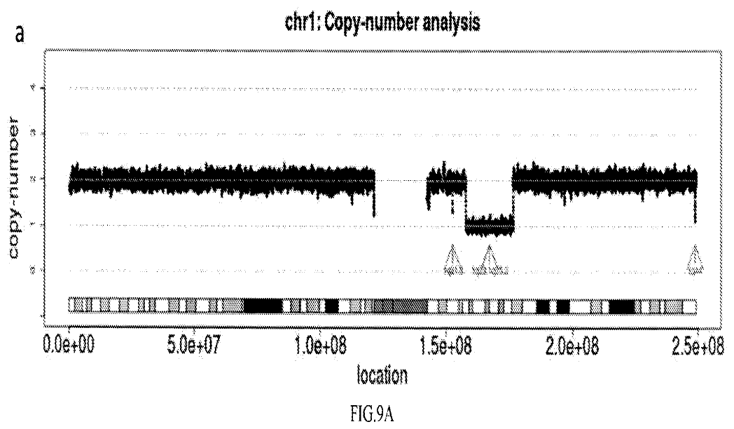

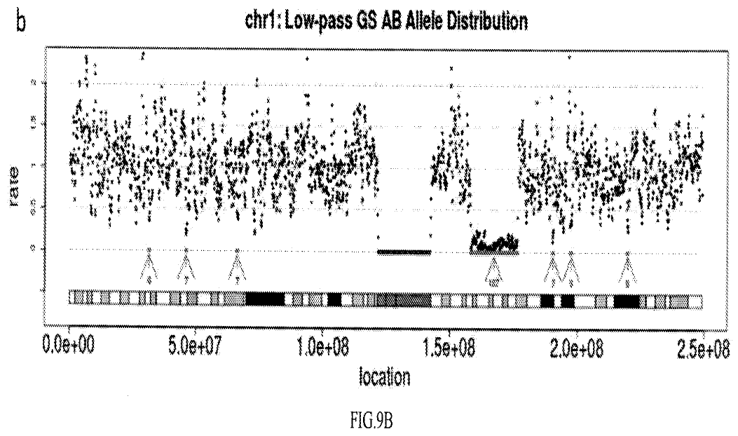

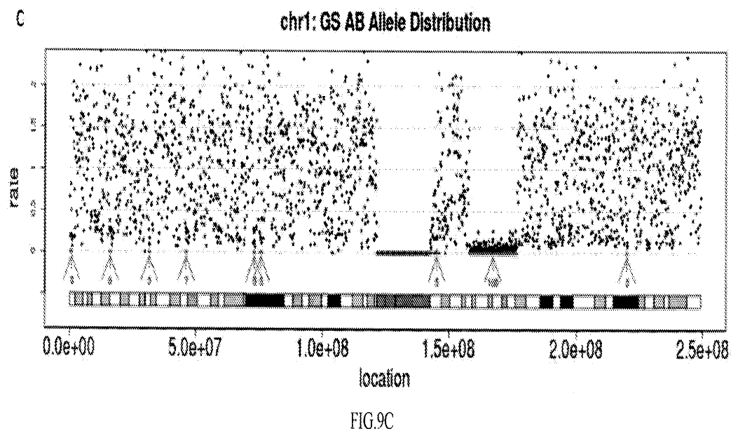

Based on the assumption that a heterozygous deletion results in copy-number loss AOH due to the absence of one allele, we further determined the cutoffs of the rates of heterozygous SNVs for AOH detection in both GS and low-pass GS based in eight cases with deletions larger than 500-kb reported from our previous study [27]. As expected, all regions with heterozygous deletions showed decreased rates of heterozygous SNVs (FIG. 9), however, the deviation of these rates detected by low-pass GS was larger than that of GS (FIG. 9). In order to reduce the deviation by low-pass GS, we further normalized each rate with the value from flanking windows for genome-widely screening of candidate regions with AOH and reported the precise boundaries by using the original values.

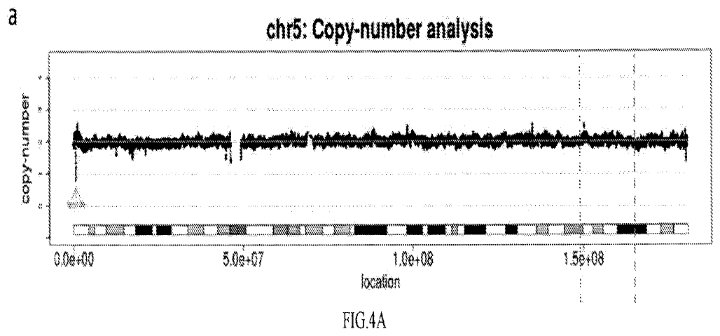

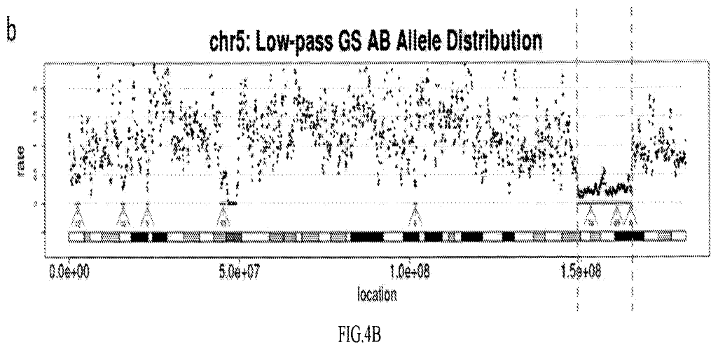

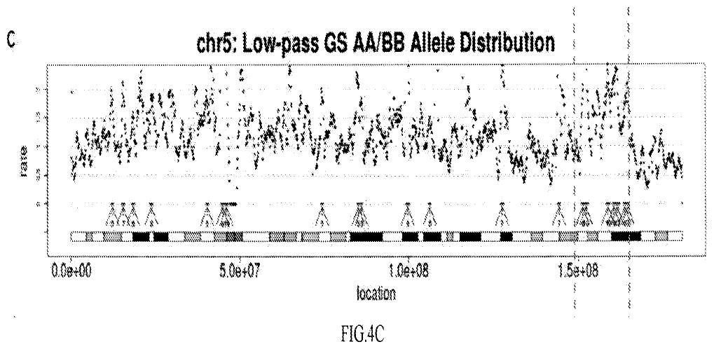

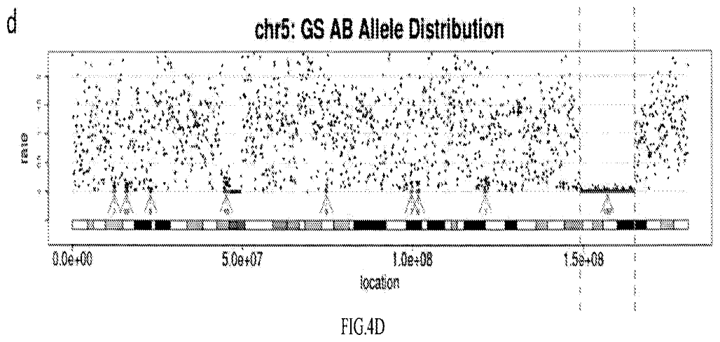

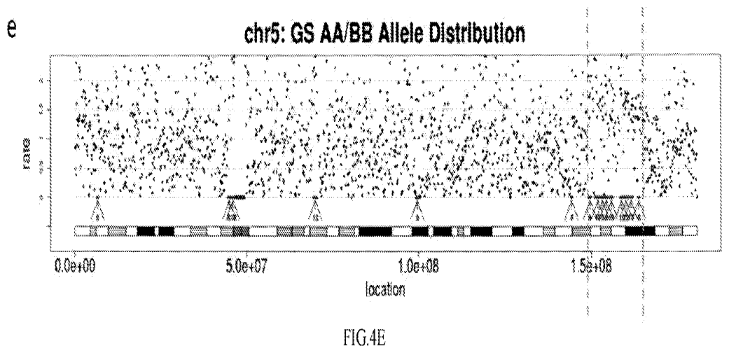

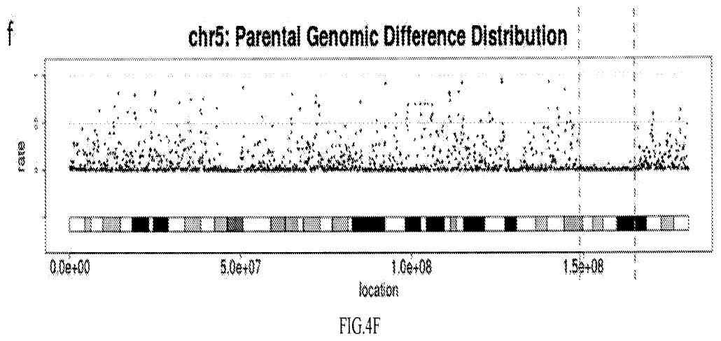

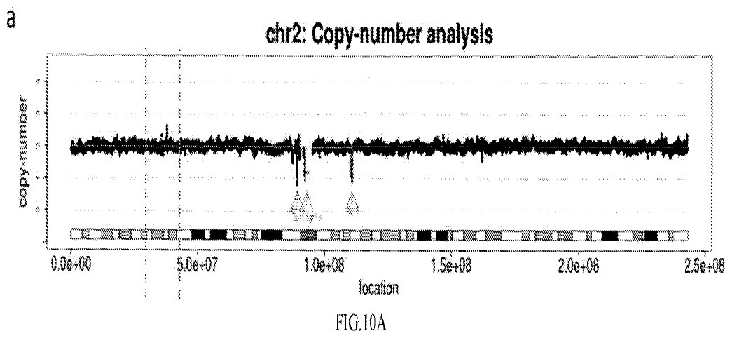

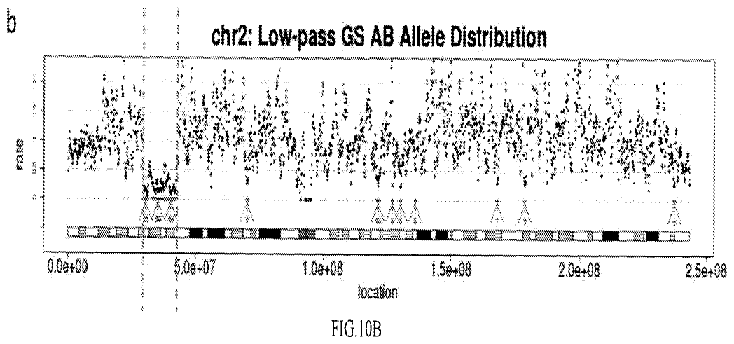

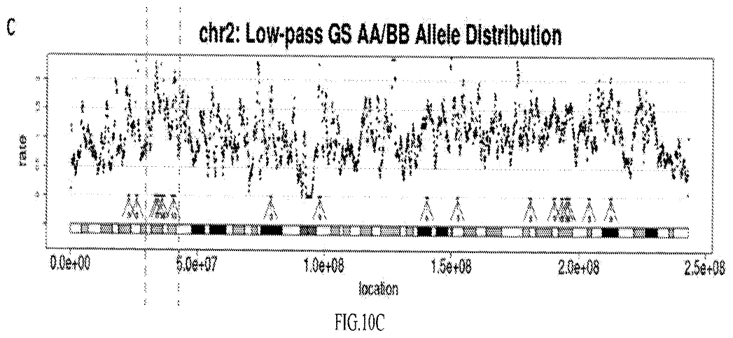

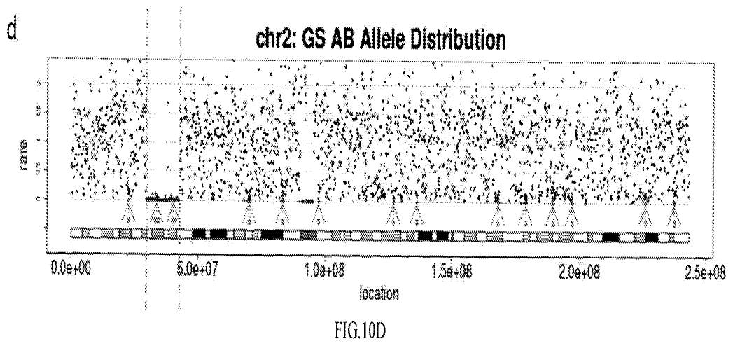

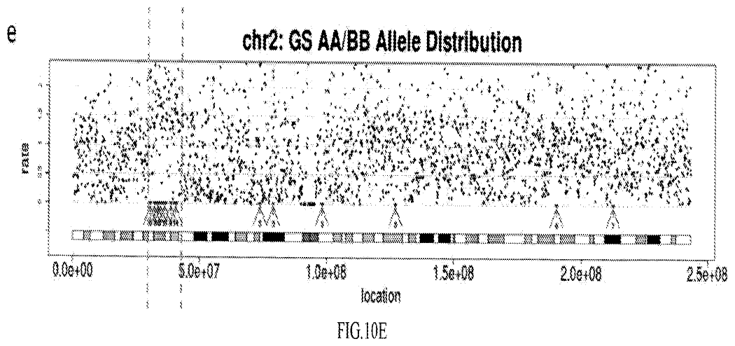

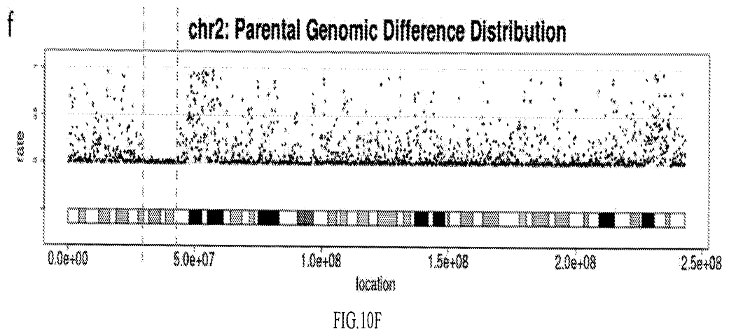

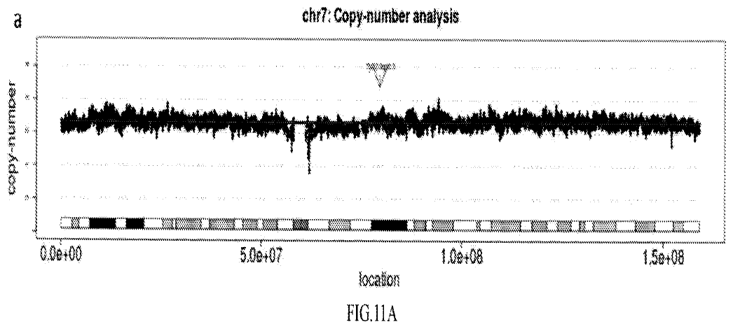

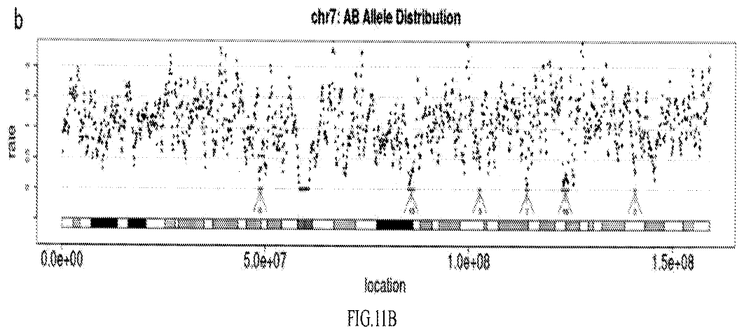

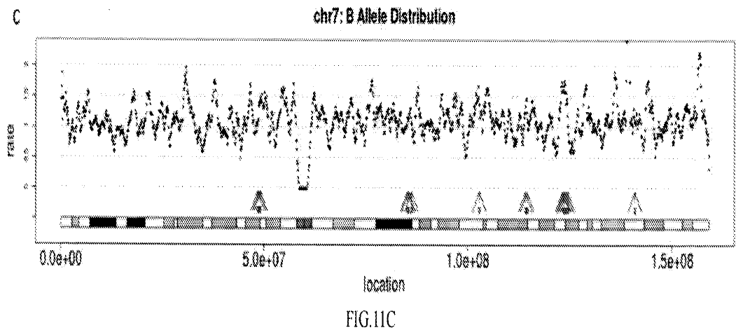

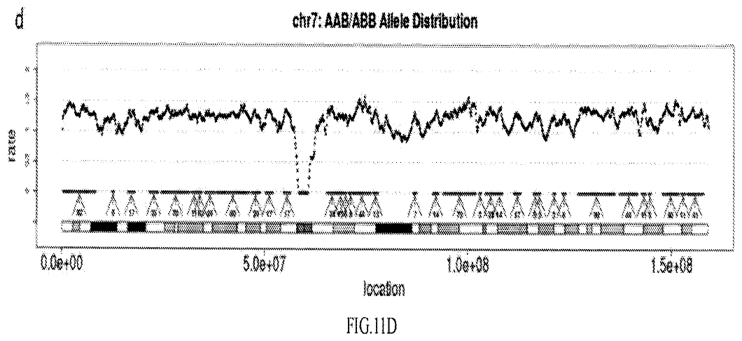

Among these cases, we incidentally identified two AOH seq[GRCh37] 2p23.2p21 (29700000_42600000)x2 hmz (FIG. 10) and seq[GRCh37] 5q23q34 (149200000_164900000)x2 hmz (FIG. 4) in HG00733, both of which were confirmed by showing the absence of parental genomic differences in those two regions. It indicated that they might be resulted from identity-by-descent (FIG. 10F and FIG. 4F). We further sought the validation by the SNP array results at a default resolution of 1-Mb, both of these two AOH were confirmed (FIG. 11). It demonstrated the reliability of the AOH detection by the observation of decreased rates of diploid heterozygous SNVs.

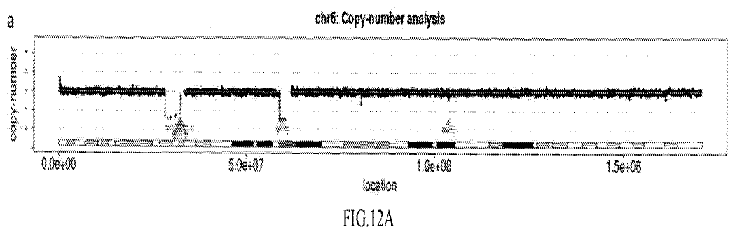

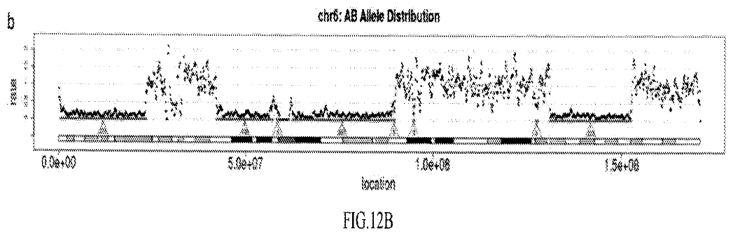

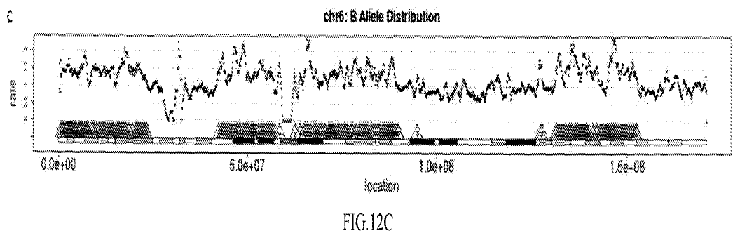

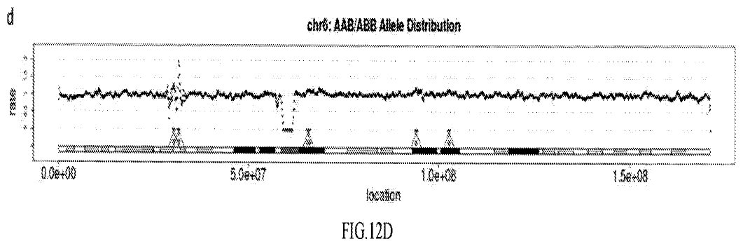

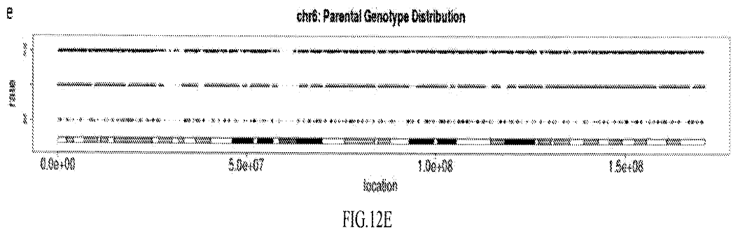

In addition, within these two regions, >50% and >25% of windows were shown to have rates of homozygous SNVs >1.25 in GS and low-pass GS, respectively. This result confirmed the observation of increased rates of homozygous SNVs when the parental genomic differences decreased (FIG. 2B). Therefore, by incorporating the rates of homozygous SNVs for further ruling out false positives, which are likely contributed by repetitive sequences such as low-copy repeats, we further compared the results generated by SNP array and by low-pass GS. In total, SNP array reported 13 AOH (>1-Mb), size of which ranging from 1.1 to 8.3-Mb, in nine regions and only in HG00733, while low-pass GS detected 87 AOH (>500-kb), including 16 AOH with 1-Mb in HG00733. All reported AOH by SNP array were consistently detected by low-pass GS. In addition, for the three additional AOH >1-Mb reported by low-pass GS, absence of heterozygous SNVs in these regions indicated the reliability of AOH detection by low-pass GS (FIG. 12).

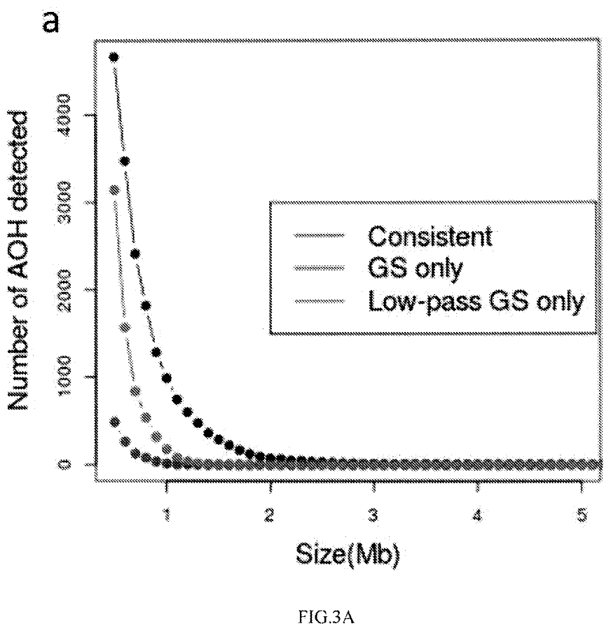

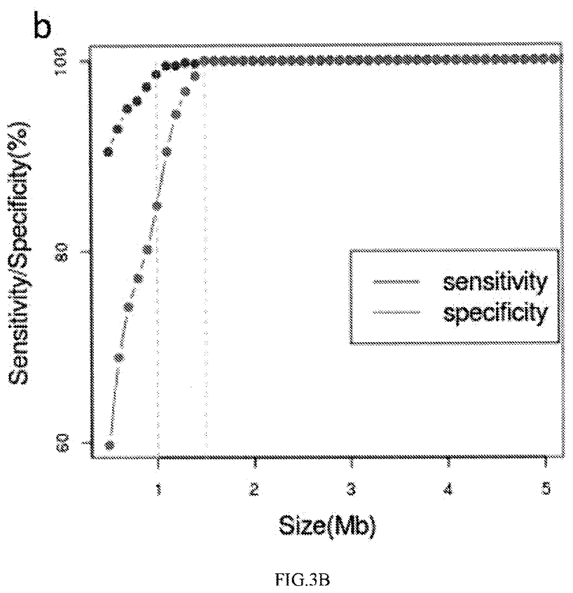

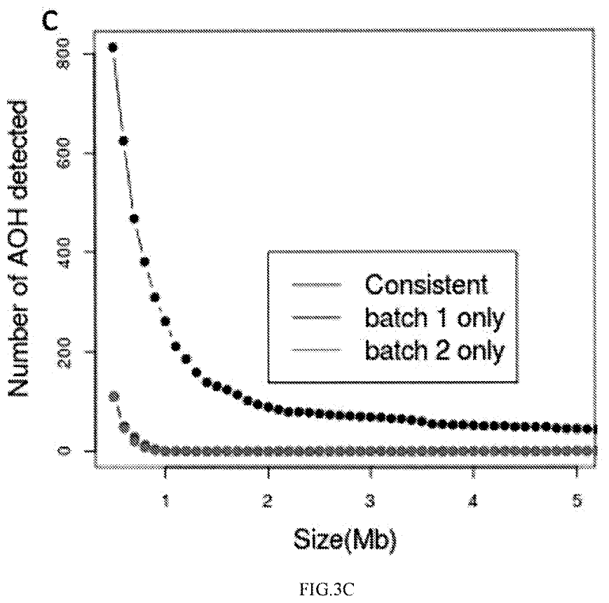

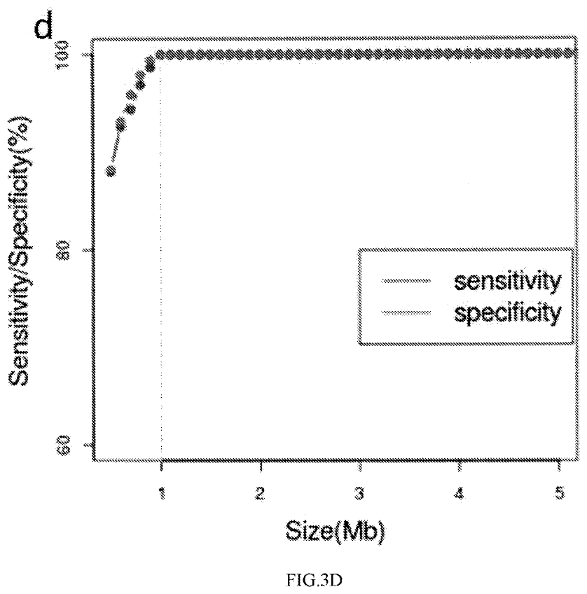

Overall, by incorporating the filtering of both rates of diploid heterozygous and homozygous SNVs, both 100% sensitivity and specificity of detecting AOH by low-pass GS were achieved when the resolution set at 1.4-Mb by using the results from GS as reference (FIGS. 3A-3B). Of note that when the resolution set as high as 1-Mb both sensitivity and specificity achieved >90.0% (FIG. 3B). 15 Validation with Clinical Samples Known to Have AOH We further applied low-pass GS for 12 clinical samples (from 10 cases) known to have multiple AOH reported by CMA (Table 1). After detection of AOH, the results showed 100% consistent with those reported by CMA (the resolution cutoff set as 5-Mb [5], Table 1). Additionally, low-pass GS was able to report additional cryptic AOH due to the lack of sufficient SNP probes in targeted regions in the CMA platform (FIG. 4). Among these cases, low-pass GS reported four cases with AOH affecting multiple chromosomes and eight cases affecting one single chromosome (Table 1). Moreover, in order to evaluate the reproducibility of this method, five of 12 samples were subjected for replication of the whole experiment including library construction, sequencing and data analysis. With the same parameters, 100% consistency between the data from the 1st batch and the replicated was achieved when the resolution set as 1.0-Mb (FIGS. 3E-3F).

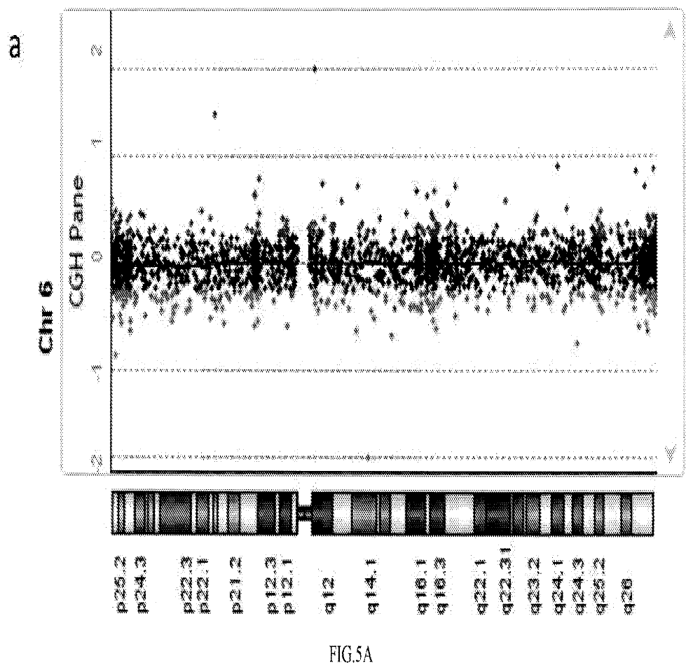

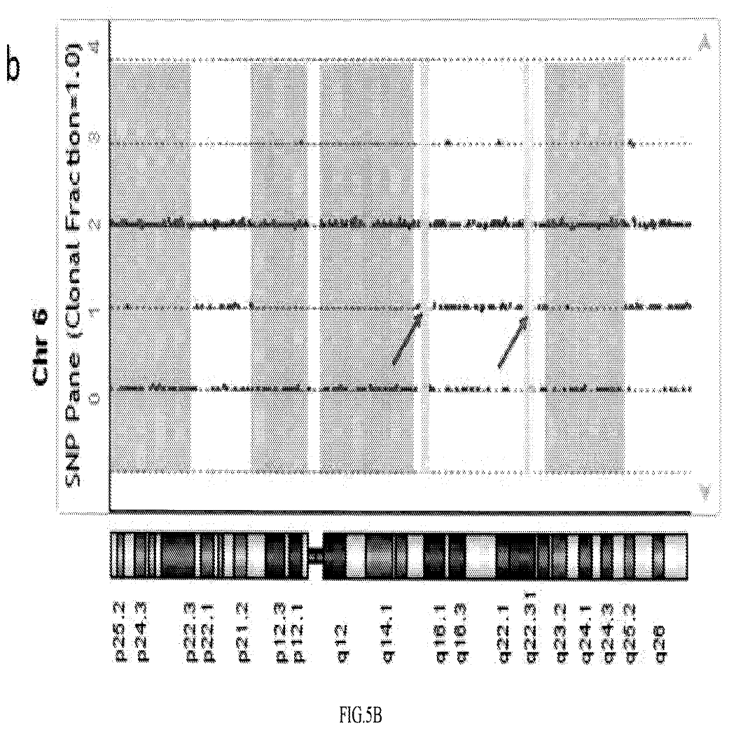

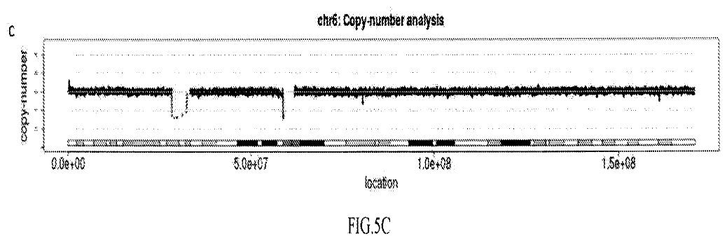

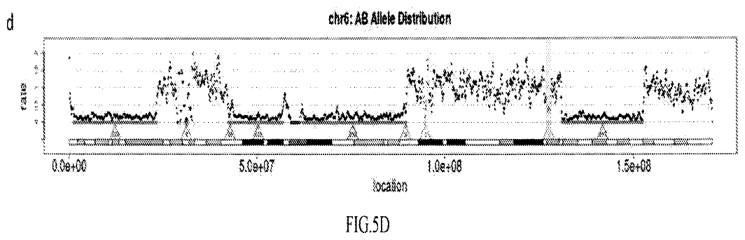

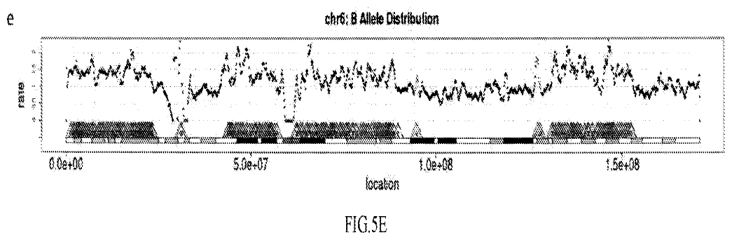

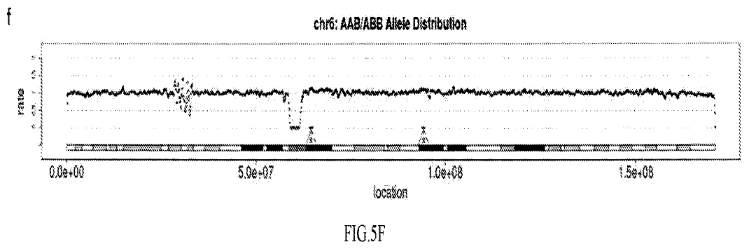

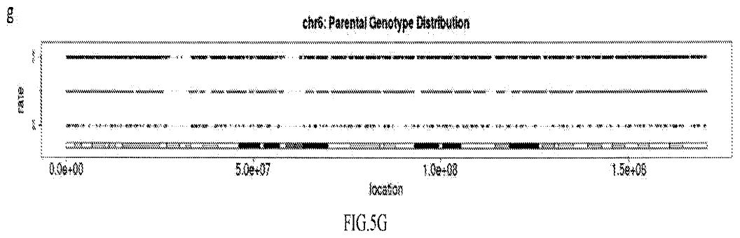

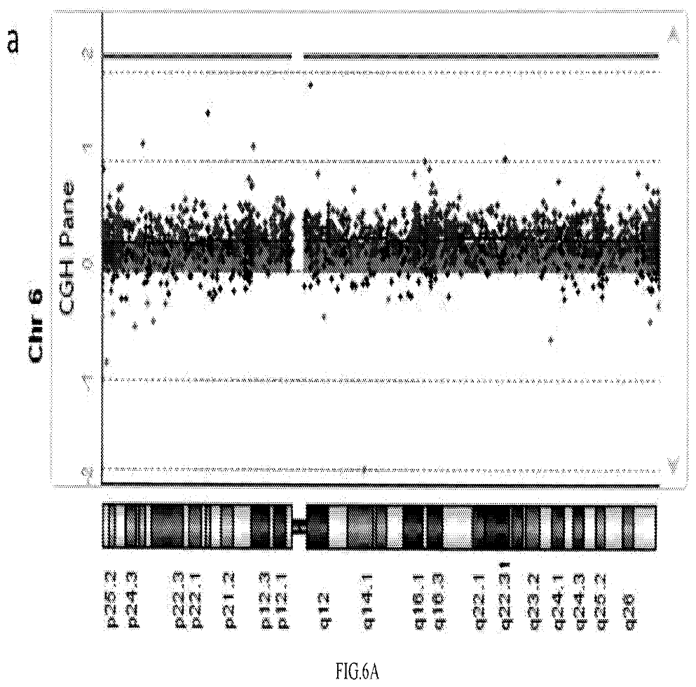

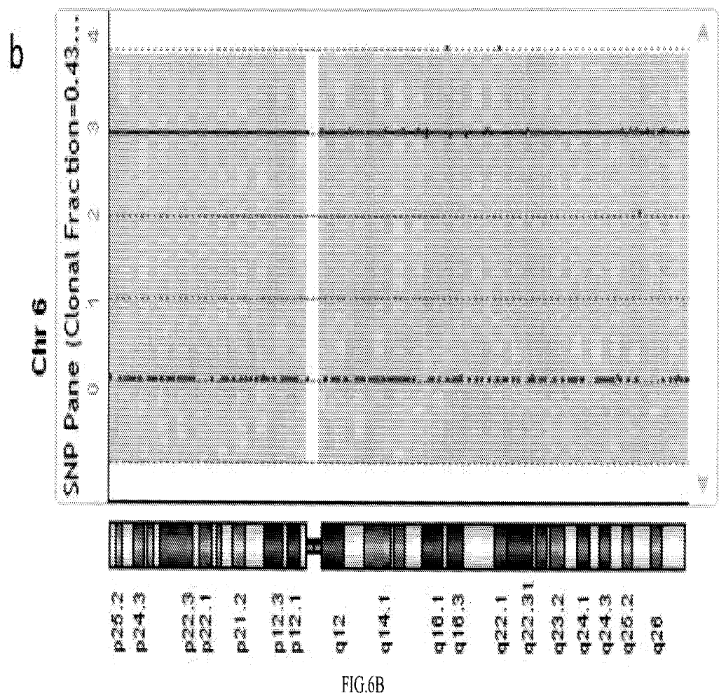

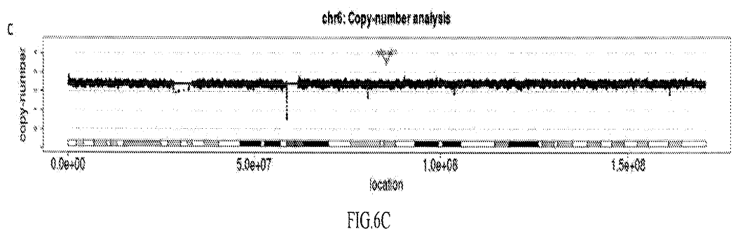

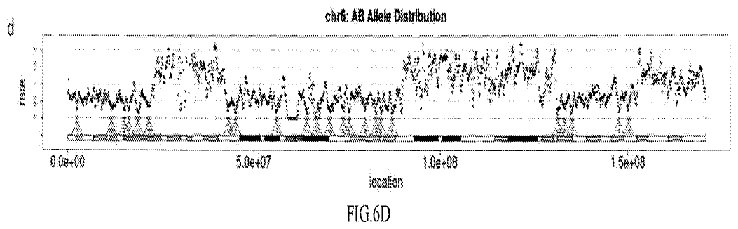

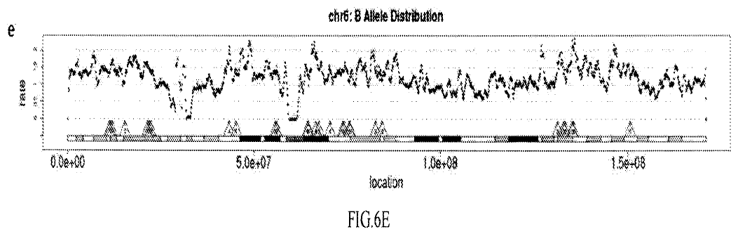

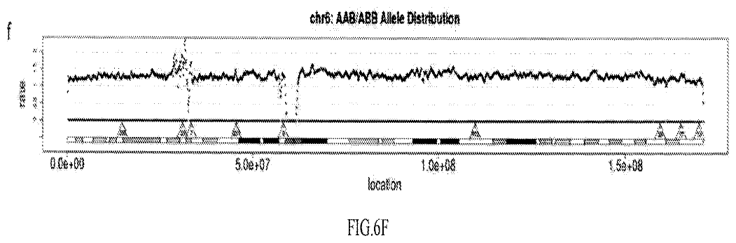

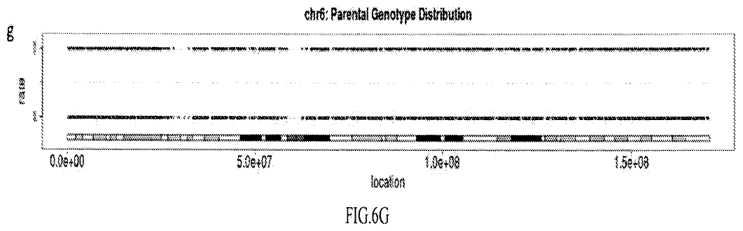

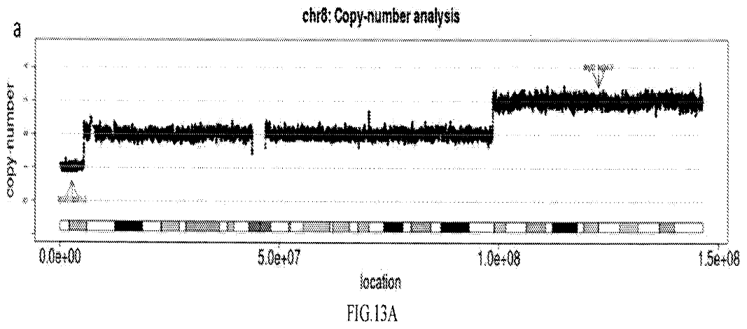

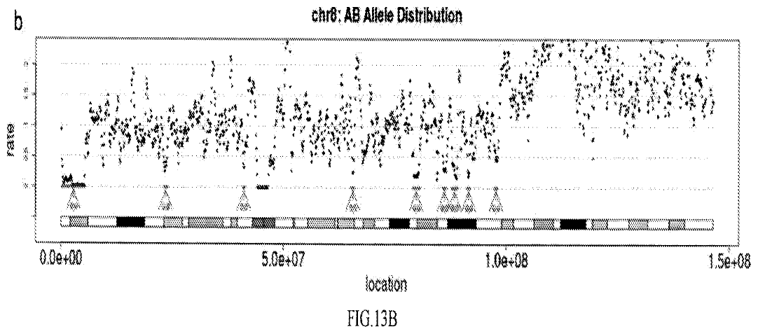

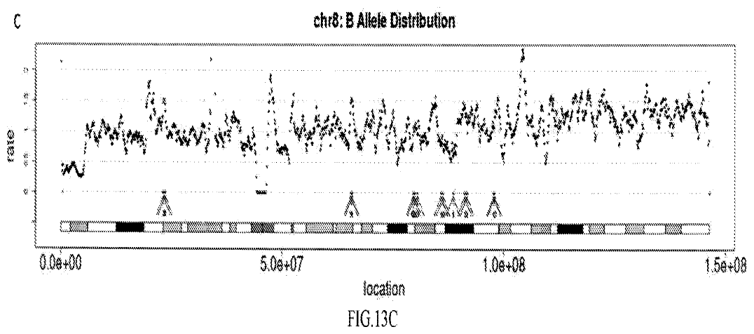

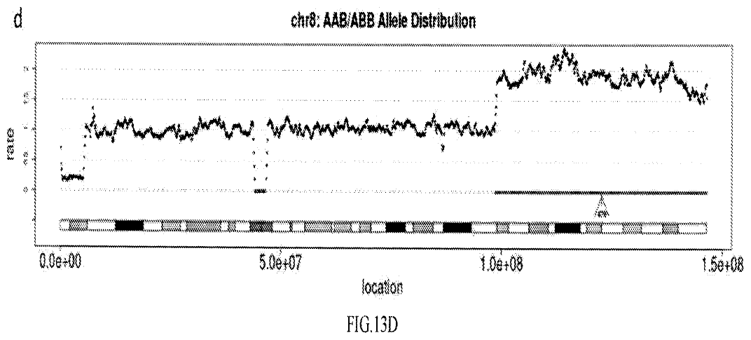

In addition, a co-occurrence of increased rates of “mosaic” heterozygous SNVs and mosaic trisomy across the whole affected chromosomes were observed in two CVS samples (FIG. 6 and FIG. 13). It indicated a biased fraction of one allele across the whole affected chromosome, consistent with the observations by CMA (FIG. 6B). Among them, the case (FIG. 6) had CVS sample 18C1493, AF (18C1564) and fetal cord blood samples (aCGH15274) available. In comparison with the results from the CVS, both AF and fetal cord blood samples showed diploid chromosomes 6 but with multiple AOH (FIG. 5) likely resulted from UPD [9]. In order to determine the presence of uniparental heterodisomy, we further performed low-pass GS for the parents. The results in both AF and fetal cord blood samples showed a pair of diploid chromosomes 6 were both of maternal origin (FIG. 6G), which was then confirmed by QF-PCR with STR markers. Therefore, the complete study in this family with multiple sample types demonstrated a case of trisomic rescue, and also demonstrated the reliability of detecting AOH by low-pass GS. In addition, the ability of determining the parental origin of each chromosome/segment by low-pass GS was further demonstrated by confirming the maternal origin of a pair of diploid chromosomes 15 in sample 16C0067.

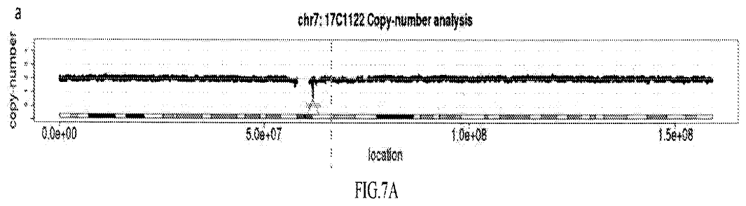

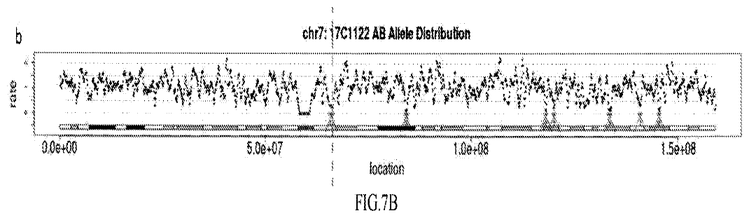

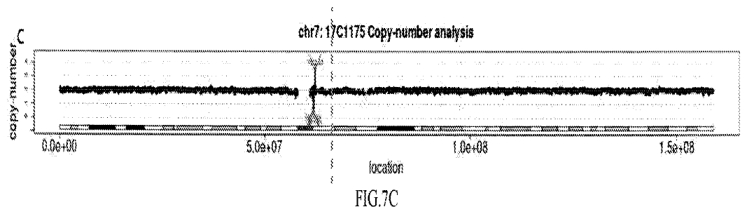

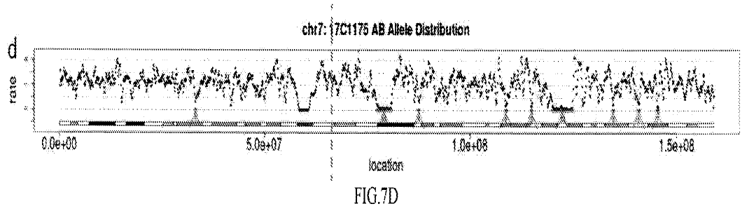

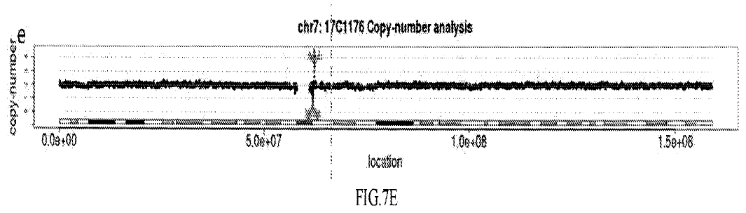

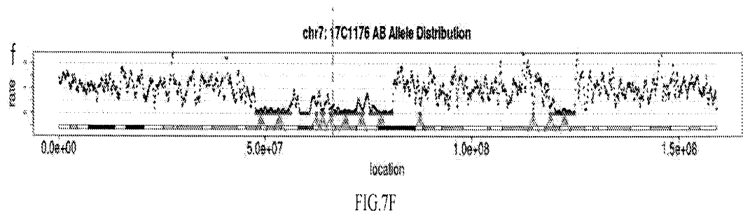

Moreover, the advantages of applying low-pass GS in pinpointing the precise boundaries and reporting cryptic AOH were further demonstrated in a consanguineous family (Table 1). Case 17C1122 with CVS was submitted for prenatal diagnosis at gestational week of 12+2 due to a family history of an elder male sibling 17C1176 diagnosed with myoclonic seizure, developmental delay, dysarthria and truncal ataxia. ES in the elder sibling identified a homozygous variants NM_153033: c50T>A in KCTD7, resulting in autosomal recessive progressive myoclonic epilepsy-3 with/without intracellular inclusion (EPM3, OMIM: 611726), while this variant was heterozygous in the unaffected sibling 17C1175. Both low-pass GS and CMA detected a region of AOH in 17C1176 seq[GRCh37] 7q11.21q11.23 (65500000_72400000)x2 hmz, encompassing KCTD7, and this AOH was absent in 17C1175 (FIG. 7). Although CMA didn't report an AOH involving KCTD7 in the fetus, Sanger sequencing at the time of prenatal diagnosis confirmed the presence of homozygous wild-type T in 17C1122. The pregnancy was continued and resulted in a normal livebirth. By blind detection, low-pass GS reported an additional 1.2-Mb AOH seq[GRCh37] 7q11.21 (65500000_66700000)x2 hmz in 17C1122 involving KCTD7 (FIG. 7), confirming the presence of homozygous wild-type Tin 17C1122 was resulted from this small AOH. We further discussed whether the genotypic information would be identified in these three cases 17C1122, 17C1175 and 17C1176, respectively. Homozygous wild type T with six supporting reads was detected in 17C1122: heterozygous base type AT with three reads was reported in 17C1175, and homozygous mutant type A with seven reads was in 17C1176. All base calls were consistent with previous Sanger sequencing results demonstrating the possibility of identifying the causative mutation(s) with precise genotypes (heterozygous/homozygous) by low-pass GS, although the read-depth coverage is not as high as that of GS. This was further demonstrated by showing the absence of a hemizygous T-C-A haplotype (with on average three supporting reads due to absence of one copy in this region) in a 16p11.2 recurrent deletion syndrome [38, 39] in low-pass GS consistent with the results provided by GS [27].

Overall, this study showed the robustness of applying low-pass GS in detection of AOH at a significantly higher resolution with precise boundaries detected and the identification of uniparental heterodisomy and isodisomy.

### Discussion

In this study, we described a robust platform-neutral method for identification of genome-wide absence of heterozygosity (AOH) by low-pass genome sequencing (GS, ˜4-fold). By comparison with GS (>30-fold) from 53 cases, our study demonstrated that both sensitivity and specificity of AOH detection with low-pass GS achieved a >90.0% at the resolution of 1-Mb and became 100% at 1.4-Mb. In addition, among 12 clinical samples with reported AOH, this method not only confirmed all known AOH and reported uniparental heterodisomy and isodisomy, but also detected additional cryptic AOH with precise genotypes provided. In the replication study, 100% consistency between the data from the 1st batch and the replicated was achieved when the resolution set as 1.0-Mb. Overall, this study demonstrated the robustness and reproducibility of this method in AOH detection.

In this study, the rates of heterozygous and homozygous SNVs were mainly utilized for detecting AOH. It was supported by the observation that parental genomic differences positively correlated with the rates of heterozygous SNVs but negatively correlated with the rates of homozygous SNVs. Moreover, the reliability of using low-pass GS for the detection was demonstrated with the high correlation of the rates of heterozygous/homozygous SNVs between GS and low-pass GS (FIGS. 2D-2E). In addition, this method not only reported 13 AOH (>1-Mb) consistently with a high-density SNP array (with a total number of 2.5 million probes), but also detected three cryptic novel AOH in a highly referenced case HG00733 [6, 26] (FIG. 12). Among a total number of 16 AOH reported by low-pass GS, there were two AOH with length larger than 10-Mb (FIG. 4 and FIG. 11). Within these two regions, there were seven and 23 OMIM disease-causing genes reported, respectively, including both autosomal dominant (i.e., SOSI) and recessive genes (i.e., CYPIBI). Although these two AOH were reported in a presumably normal individual, involvement of OMIM disease-causing genes emphases the importance of AOH detection and this finding indicates the importance of defining the spectrum of AOH by using the data from the presumably normal individuals such as from the 1000 Genomes Project.

Validation with 12 clinical samples with multiple AOH reported by CMA, we further demonstrated the 100% consistency of AOH detection between low-pass GS and routine CMA (5-Mb, the maximum resolution of the CMA platform used). Furthermore, the importance of detecting AOH at a higher resolution was demonstrated by the identification of a cryptic 1.2-Mb AOH in a prenatal case involving KCTD7, a homozygous variant located in which caused the severe phenotypes in the elder sibling due to the presence of a large segment of AOH. In addition, low-pass GS also showed the possibility of providing precise genotypes among this family (the fetus and the two elder siblings) and in the hemizygous allele of the 16p11.22 recurrent deletion syndrome, although the number of supporting reads was limited. Based on this increased resolution, we are able to identify those critical regions known to carry the imprinted genes such as the 2-Mb domain on chromosome 15q11-q13 affecting the Prade-willi and Angelman syndromes [40]. In addition, for the two cases with parental low-pass GS results were available, we demonstrated the feasibility of determining the parental origin using the genotypic information supported by the limited read-depths. With such information, we were able to identify uniparental heterodisomy (without AOH) in the affected chromosomes with the presence of uniparental isodisomy (AOH, FIG. 5G).

This method is sequencing platform neutral (applicable in data generated from Illumina and MGI) and irrespective of sequencing read-lengths (126-bp in the data downloaded from the 1000 Genomes Project and 100-bp in the data sequenced in present study), providing the possibility of incorporating this test into the sequencing runs for ES or GS. Currently, many laboratories provide GS/ES testing with paired-end 150-bp sequencing, the number of read-pairs required to reach ˜4-fold coverage required for the AOH analysis can be set as low as 40 million indicating this would be one of the most cost-efficient tests.

Overall, this study shows the reliability of using the combination of the rates of “germline”/“mosaic” heterozygous and homozygous SNVs for the identification of germline and mosaic AOH. For example, a combination of decreased rates of “germline” heterozygous SNVs and increased rate of homozygous SNVs were used for the identification of AOH. Furthermore, combination of different parameters would assist CNV detection. For example, all rates decreased resulted from a heterozygous deletion, or the increased rates of “mosaic” heterozygous SNVs in a region with duplication.

### CONCLUSION

This study describes a robust method for detecting AOH by utilizing low-pass GS (with ˜4-fold) at a significant higher resolution compared to routine CMAs and even high-density SNP array. In addition, by showing a significant high consistency of AOH detection with low-pass GS compared with the results reported by GS and CMA, our study provides compelling evidence to implement this method for AOH detection in the context of utilizing low-pass GS for routine genetic testing.

### REFERENCES

1. Karampetsou E, Morrogh D, Chitty L: Microarray Technology for the Diagnosis of Fetal Chromosomal Aberrations: Which Platform Should We Use? J Clin Med 2014, 3 (2): 663-678.
2. Liu S, Zhang K, Song F, Yang Y, Lv Y, Gao M, Liu Y, Gai Z: Uniparental Disomy of Chromosome 15 in Two Cases by Chromosome Microarray: A Lesson Worth Thinking. Cytogenet Genome Res 2017, 152 (1): 1-8.
3. Margraf R L, VanSant-Webb C, Sant D, Carey J, Hanson H, D'Astous J, Viskochil D, Stevenson D A, Mao R: Utilization of Whole-Exome Next-Generation Sequencing Variant Read Frequency for Detection of Lesion-Specific, Somatic Loss of Heterozygosity in a Neurofibromatosis Type 1 Cohort with Tibial Pseudarthrosis. J Mol Diagn 2017, 19 (3): 468-474.
4. Liu X, Li A, Xi J, Feng H, Wang M: Detection of copy number variants and loss of heterozygosity from impure tumor samples using whole exome sequencing data. Oncol Lett 2018, 16 (4): 4713-4720.
5. D'Amours G, Langlois M, Mathonnet G, Fetni R, Nizard S, Srour M, Tihy F, Phillips M S, Michaud J L, Lemyre E: SNP arrays: comparing diagnostic yields for four platforms in children with developmental delay. BMC Med Genomics 2014, 7:70.
6. Dharmadhikari A V, Ghosh R, Yuan B, Liu P, Dai H, Al Masri S, Scull J, Posey J E, Jiang A H, He W et al: Copy number variant and runs of homozygosity detection by microarrays enabled more precise molecular diagnoses in 11,020 clinical exome cases. Genome Med 2019, 11 (1): 30.
7. Robinson W P: Mechanisms leading to uniparental disomy and their clinical consequences. Bioessays 2000, 22 (5): 452-459.
8. Eggermann T, Soellner L, Buiting K, Kotzot D: Mosaicism and uniparental disomy in prenatal diagnosis. Trends Mol Med 2015, 21 (2): 77-87.
9. Conlin L K, Thiel B D, Bonnemann C G, Medne L, Ernst L M, Zackai E H, Deardorff M A, Krantz I D, Hakonarson H, Spinner N B: Mechanisms of mosaicism, chimerism and uniparental disomy identified by single nucleotide polymorphism array analysis. Hum Mol Genet 2010, 19 (7): 1263-1275.
10. Fridman C, Koiffmann CP: Origin of uniparental disomy 15 in patients with Prader-Willi or Angelman syndrome. Am J Med Genet 2000, 94 (3): 249-253.
11. Normand E A, Braxton A, Nassef S, Ward P A, Vetrini F, He W, Patel V, Qu C, Westerfield L E, Stover S et al: Clinical exome sequencing for fetuses with ultrasound abnormalities and a suspected Mendelian disorder. Genome Med 2018, 10 (1): 74.
12. Drury S, Williams H, Trump N, Boustred C, Gosgene, Lench N, Scott R H, Chitty LS: Exome sequencing for prenatal diagnosis of fetuses with sonographic abnormalities. Prenat Diagn 2015, 35 (10): 1010-1017.
13. Leung G K C, Mak C C Y, Fung J L F, Wong W H S, Tsang M H Y, Yu M H C, Pei S L C, Yeung K S, Mok G T K, Lee C P et al: Identifying the genetic causes for prenatally diagnosed structural congenital anomalies (SCAs) by whole-exome sequencing (WES). BMC Med Genomics 2018, 11 (1): 93.
14. Lord J, McMullan D J, Eberhardt R Y, Rinck G, Hamilton S J, Quinlan-Jones E, Prigmore E, Keelagher R, Best S K, Carey G K et al: Prenatal exome sequencing analysis in fetal structural anomalies detected by ultrasonography (PAGE): a cohort study. Lancet 2019, 393 (10173): 747-757.
15. Petrovski S, Aggarwal V, Giordano J L, Stosic M, Wou K, Bier L, Spiegel E, Brennan K, Stong N, Jobanputra V et al: Whole-exome sequencing in the evaluation of fetal structural anomalies: a prospective cohort study. Lancet 2019, 393 (10) 173): 758-767.
16. Fu F, Li R, Li Y, Nie Z Q, Lei T, Wang D, Yang X, Han J, Pan M, Zhen L et al: Whole exome sequencing as a diagnostic adjunct to clinical testing in fetuses with structural abnormalities. Ultrasound Obstet Gynecol 2018, 51 (4): 493-502.
17. Sathirapongsasuti J F, Lee H, Horst B A, Brunner G, Cochran A J, Binder S, Quackenbush J, Nelson S F: Exome sequencing-based copy-number variation and loss of heterozygosity detection: ExomeCNV. Bioinformatics 2011, 27 (19): 2648-2654.
18. San Lucas F A, Sivakumar S, Vattathil S, Fowler J, Vilar E, Scheet P: Rapid and powerful detection of subtle allelic imbalance from exome sequencing data with hapLOHseq. Bioinformatics 2016, 32 (19): 3015-3017.
19. Belkadi A, Bolze A, Itan Y, Cobat A, Vincent Q B, Antipenko A, Shang L, Boisson B, Casanova J L, Abel L: Whole-genome sequencing is more powerful than whole-exome sequencing for detecting exome variants. Proc Natl Acad Sci USA 2015, 112 (17): 5473-5478.
20. Li X, Chen S, Xie W, Vogel I, Choy K W, Chen F, Christensen R, Zhang C, Ge H, Jiang H et al: PSCC: sensitive and reliable population-scale copy number variation detection method based on low coverage sequencing. PLOS One 2014, 9 (1):e85096.
21. Liang D, Peng Y, Lv W, Deng L, Zhang Y, Li H, Yang P, Zhang J, Song Z, Xu G et al: Copy number variation sequencing for comprehensive diagnosis of chromosome disease syndromes. J Mol Diagn 2014, 16 (5): 519-526.
22. Dong Z, Zhang J, Hu P, Chen H, Xu J, Tian Q, Meng L, Ye Y, Wang J, Zhang M et al: Low-pass whole-genome sequencing in clinical cytogenetics: a validated approach. Genet Med 2016, 18 (9): 940-948.
23. Dong Z, Wang H, Chen H, Jiang H, Yuan J, Yang Z, Wang W J, Xu F, Guo X, Cao Y et al: Identification of balanced chromosomal rearrangements previously unknown among participants in the 1000 Genomes Project: implications for interpretation of structural variation in genomes and the future of clinical cytogenetics. Genet Med 2018, 20 (7): 697-707.
24. Dong Z, Jiang L, Yang C, Hu H, Wang X, Chen H, Choy K W, Hu H, Dong Y, Hu B et al: A robust approach for blind detection of balanced chromosomal rearrangements with whole-genome low-coverage sequencing. Hum Mutat 2014, 35 (5): 625-636.
25. Redin C, Brand H, Collins R L, Kammin T, Mitchell E, Hodge J C, Hanscom C, Pillalamarri V, Seabra C M, Abbott M A et al: The genomic landscape of balanced cytogenetic abnormalities associated with human congenital anomalies. Nat Genet 2017, 49 (1): 36-45.
26. Chaisson M J P, Sanders A D, Zhao X, Malhotra A, Porubsky D, Rausch T, Gardner E J, Rodriguez O L, Guo L, Collins R L et al: Multi-platform discovery of haplotype-resolved structural variation in human genomes. Nat Commun 2019, 10 (1): 1784.
27. Choy K W, Wang H, Shi M, Chen J, Yang Z, Zhang R, Yan H, Wang Y, Chen S, Chau M H K et al: Prenatal Diagnosis of Fetuses with Increased Nuchal Translucency by Genome Sequencing Analysis. bioRxiv 2019:667311.
28. Leung T Y, Vogel I, Lau T K, Chong W, Hyett J A, Petersen O B, Choy K W: Identification of submicroscopic chromosomal aberrations in fetuses with increased nuchal translucency and apparently normal karyotype. Ultrasound Obstet Gynecol 2011, 38 (3): 314-319.
29. Huang J, Poon L C, Akolekar R, Choy K W, Leung T Y, Nicolaides K H: Is high fetal nuchal translucency associated with submicroscopic chromosomal abnormalities on array CGH? Ultrasound Obstet Gynecol 2014, 43 (6): 620-624.
30. Huang J, Liang X, Xuan Y, Geng C, Li Y, Lu H, Qu S, Mei X, Chen H, Yu T et al: A reference human genome dataset of the BGISEQ-500 sequencer. Gigascience 2017, 6 (5): 1-9.
31. Li H. Durbin R: Fast and accurate short read alignment with Burrows-Wheeler transform. Bioinformatics 2009, 25 (14): 1754-1760.
32. Li H, Handsaker B, Wysoker A, Fennell T, Ruan J, Homer N, Marth G, Abecasis G, Durbin R, Genome Project Data Processing S: The Sequence Alignment/Map format and SAMtools. Bioinformatics 2009, 25 (16): 2078-2079.
33. McKenna A, Hanna M, Banks E, Sivachenko A, Cibulskis K, Kernytsky A, Garimella K, Altshuler D, Gabriel S, Daly M et al: The Genome Analysis Toolkit: a MapReduce framework for analyzing next-generation DNA sequencing data. Genome Res 2010, 20 (9): 1297-1303.
34. Wang K, Li M, Hakonarson H: ANNOVAR: functional annotation of genetic variants from high-throughput sequencing data. Nucleic Acids Res 2010, 38 (16):e164.
35. Dong Z, Xie W, Chen H, Xu J, Wang H, Li Y, Wang J, Chen F, Choy K W, Jiang H: Copy-Number Variants Detection by Low-Pass Whole-Genome Sequencing. Curr Protoc Hum Genet 2017, 94:8 17 11-18 17 16.
36. Cheng Y K, Wong C, Wong H K, Leung K O, Kwok Y K, Suen A, Wang C C, Leung T Y, Choy K W: The detection of mosaicism by prenatal BoBs, Prenat Diagn 2013, 33 (1): 42-49.
37. Delaneau O, Marchini J, Genomes Project C, Genomes Project C: Integrating sequence and array data to create an improved 1000 Genomes Project haplotype reference panel. Nat Commun 2014, 5:3934.
38. Wu N, Ming X, Xiao J, Wu Z, Chen X, Shinawi M, Shen Y, Yu G, Liu J, Xie H et al: TBX6 null variants and a common hypomorphic allele in congenital scoliosis. N Engl J Med 2015, 372 (4): 341-350).
39. Liu J, Wu N, Deciphering Disorders Involving S, study C O, Yang N, Takeda K, Chen W, Li W, Du R, Liu S et al: TBX6-associated congenital scoliosis (TACS) as a clinically distinguishable subtype of congenital scoliosis: further evidence supporting the compound inheritance and TBX6 gene dosage model. Genet Med 2019.
40. Perk J, Makedonski K, Lande L, Cedar H, Razin A, Shemer R: The imprinting mechanism of the Prader-Willi/Angelman regional control center. EMBO J 2002, 21 (21): 5807-5814.

What is claimed is:

1. A method of detecting absence of heterozygosity (AOH) in a biological sample from a subject, comprising
(i) receiving sequence reads from low-pass genome sequencing of genomic DNA of the biological sample;
(ii) aligning the sequence reads to a human genome reference, and selecting and sorting sequence reads aligned to the human genome reference based on the aligned chromosome and genomic coordinates;
(iii) identifying single-nucleotide variants (SNVs) in the aligned sequence reads, wherein a single-nucleotide variant at each site has a mutant base type different from the base type at the corresponding site from the human genome reference;
(iv) identifying homozygous SNVs, diploid heterozygous SNVs, or non-diploid heterozygous SNVs from the SNVs identified in step (iii), wherein
a homozygous SNV is define based on the percentage of sequence reads supporting the mutant base type different from the base type at the corresponding site from the human genome reference being 100%,
a diploid heterozygous SNV is define based on the percentage of sequence reads supporting the mutant base type different from the base type at the corresponding site from the human genome reference being no less than 25% and no large than 75%,
a non-diploid heterozygous SNV is define based on the percentage of sequence reads supporting the mutant base type different from the base type at the corresponding site from the human genome reference being less than 25% and larger than 0% or larger than 75% and less than 100%;
(v) determining a rate of homozygous SNVs, diploid heterozygous SNVs, or non-diploid heterozygous SNVs identified in step (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) comparing the rate of homozygous SNVs, diploid heterozygous SNVs, or non-diploid heterozygous SNVs for individual windows determined from step (v) with an average rate of homozygous SNVs, diploid heterozygous SNVs, or non-diploid heterozygous SNVs for corresponding individual windows established from a control population.

2. The method of claim 1, wherein the biological sample is selected from the group consisting of peripheral blood, chorionic villus, amniotic fluid, cord blood, placental tissue, and tissue samples from organs.

3. The method of claim 1, wherein the subject is a pregnant female, an infant, a subject suffering from a cancer, or a subject suspected of suffering from a cancer.

4. The method of claim 1, wherein the sequence reads are single-end sequence reads or paired-end sequence reads.

5. The method of claim 1, wherein the low-pass genome sequencing has a read depth of 3˜5 folds.

6. The method of claim 1, wherein the absence of heterozygosity (AOH) is copy-number neutral loss of heterozygosity (CN-LOH).

7. The method of claim 1, wherein step (ii) further includes removing sequence reads due to polymerase chain reaction (PCR) duplication.

8. The method of claim 1, wherein step (iii) further includes discarding a site as described below:
(a) a minimal read-depth of the site is determined by the minimal read-depth of the biological sample;
(b) a maximum read-depth of the site is determined by the maximal read-depth of the biological sample; or
(c) a site where no sequence read supports a mutant base type.

9. The method of claim 1, wherein the window in step (v) has a fixed length of 100-kb.

10. The method of claim 1, wherein step (v) comprises
determining the number of homozygous SNVs, diploid heterozygous SNVs, or non-diploid heterozygous SNVs for the window,
determining the average number of homozygous SNVs, diploid heterozygous SNVs, or non-diploid heterozygous SNVs among all windows in the biological sample, and
calculating the rate of homozygous SNVs, diploid heterozygous SNVs, or non-diploid heterozygous SNVs for the window by dividing the number of homozygous SNVs, diploid heterozygous SNVs, or non-diploid heterozygous SNVs identified for the window by the average number of homozygous SNVs, diploid heterozygous SNVs, or non-diploid heterozygous SNVs among all windows in the biological sample.

11. The method of claim 1, wherein the control population has the same gender as the subject.

12. The method of claim 1, wherein step (vi) comprises
normalizing the rate of homozygous SNVs, diploid heterozygous SNVs, or non-diploid heterozygous SNVs for a window by an average rate of homozygous SNVs, diploid heterozygous SNVs, or non-diploid heterozygous SNVs for the corresponding window established from the control population, thereby providing a corresponding rate ratio of homozygous SNVs, diploid heterozygous SNVs, or non-diploid heterozygous SNVs for the window.

13. The method of claim 12, wherein in step (vi), decreased rate of diploid heterozygous SNVs and increased rate of homozygous SNVs indicate AOH, and step (vi) further comprises
where the copy-number neutral expressing as copy-ratios equal to 1, for all windows with diploid heterozygous SNVs rate ratios less than 1, defining a region if there are a plurality of windows with consecutive diploid heterozygous SNVs rate ratios less than 0.5 and the percentage of windows with homozygous rate ratios larger than 1.25 is at least 30%, and optionally combining two regions into one if there are no more than one windows with diploid heterozygous SNVs rate ratios larger than 0.5 but less than 1; and
reporting the region as presence of AOH.

14. The method of claim 12, wherein in step (vi), increased rate of non-diploid heterozygous SNVs indicates mosaic AOH, and step (vi) further comprises
where the copy-number mosaic duplication represented as copy-ratio larger than 1, for all windows with non-diploid heterozygous SNVs rate ratios larger than 1, defining a region if there are a plurality of windows with consecutive non-diploid heterozygous SNVs rate ratios larger than 1.15; and
reporting the region as presence of mosaic AOH.

15. The method of claim 1, wherein an average rate of heterozygous SNVs for corresponding individual windows established from a control population is determined by
(ci) receiving sequence reads from low-pass genome sequencing of genomic DNA of a biological sample from a control subject from the control population;
(cii) aligning the sequence reads to a human genome reference, and selecting and sorting sequence reads aligned to the human genome reference based on the aligned chromosome and genomic coordinates;
(ciii) identifying single-nucleotide variants (SNVs) in the aligned sequence reads, wherein a single-nucleotide variant at each site has a mutant base type different from the base type at the corresponding site from the human genome reference;
(civ) identifying homozygous SNVs, diploid heterozygous SNVs, or non-diploid heterozygous SNVs from the SNVs identified in step (ciii), wherein
a homozygous SNV is define based on the percentage of sequence reads supporting the mutant base type different from the base type at the corresponding site from the human genome reference being 100%,
a diploid heterozygous SNV is define based on the percentage of sequence reads supporting the mutant base type different from the base type at the corresponding site from the human genome reference being no less than 25% and no large than 75%,
a non-diploid heterozygous SNV is define based on the percentage of sequence reads supporting the mutant base type different from the base type at the corresponding site from the human genome reference being less than 25% and larger than 0% or larger than 75% and less than 100%;
(cv) determining a rate of homozygous SNVs, diploid heterozygous SNVs, or non-diploid heterozygous SNVs identified in step (civ) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample from the control subject; and
(cvi) averaging rates of homozygous SNVs, diploid heterozygous SNVs, or non-diploid heterozygous SNVs for a window from all control subjects to provide an average rate of homozygous SNVs, diploid heterozygous SNVs, or non-diploid heterozygous SNVs for the corresponding window in the control population.

16. The method of claim 15, further comprising, between step (cii) and (ciii), a step of sex determination, wherein the aligned ratios of chromosome X, chromosome Y and the whole genome are calculated as the numbers of sequence reads aligned to the chromosome/genome dividing by the length defined by the humane reference genome, respectively, the chromosome Y percentage is calculated as the aligned ratio of chromosome Y dividing by the aligned ratio of the whole genome, and a control subject is considered as male if the chromosome Y percentage is larger than 0.1.

17. The method of claim 16, wherein steps (ciii) to (cvi) are carried out on male and female control subjects respectively, based on the result of the step of sex determination.

18. The method of claim 15, wherein, in step (cvi), if rates of homozygous SNVs, diploid heterozygous SNVs, or non-diploid heterozygous SNVs for a window among control subjects have substantial deviation, the average rate of homozygous SNVs, diploid heterozygous SNVs, or non-diploid heterozygous SNVs for the window is calculated as an average of the rates of the window and its flanking windows.

19. A computer readable medium storing a plurality of instructions, wherein the plurality of instructions, upon executed by one or more processors, perform an operation including
(i) receiving sequence reads from low-pass genome sequencing of genomic DNA of a biological sample from a subject;
(ii) aligning the sequence reads to a human genome reference, and selecting and sorting sequence reads aligned to the human genome reference based on the aligned chromosome and genomic coordinates;
(iii) identifying single-nucleotide variants (SNVs) in the aligned sequence reads, wherein a single-nucleotide variant at each site has a mutant base type different from the base type at the corresponding site from the human genome reference;
(iv) identifying homozygous SNVs, diploid heterozygous SNVs, or non-diploid heterozygous SNVs from the SNVs identified in (iii), wherein
a homozygous SNV is define based on the percentage of sequence reads supporting the mutant base type different from the base type at the corresponding site from the human genome reference being 100%,
a diploid heterozygous SNV is define based on the percentage of sequence reads supporting the mutant base type different from the base type at the corresponding site from the human genome reference being no less than 25% and no large than 75%,
a non-diploid heterozygous SNV is define based on the percentage of sequence reads supporting the mutant base type different from the base type at the corresponding site from the human genome reference being less than 25% and larger than 0% or larger than 75% and less than 100%;
(v) determining a rate of homozygous SNVs, diploid heterozygous SNVs, or non-diploid heterozygous SNVs identified in (iv) for a window; wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) comparing the rate of homozygous SNVs, diploid heterozygous SNVs, or non-diploid heterozygous SNVs for individual windows determined from (v) with an average rate of homozygous SNVs, diploid heterozygous SNVs, or non-diploid heterozygous SNVs for corresponding individual windows established from a control population.

20. A device comprising one or more processors and a computer readable medium storing a plurality of instructions, wherein the plurality of instructions, upon executed by one or more processors, perform an operation including
(i) receiving sequence reads from low-pass genome sequencing of genomic DNA of a biological sample from a subject;
(ii) aligning the sequence reads to a human genome reference, and selecting and sorting sequence reads aligned to the human genome reference based on the aligned chromosome and genomic coordinates;
(iii) identifying single-nucleotide variants (SNVs) in the aligned sequence reads, wherein a single-nucleotide variant at each site has a mutant base type different from the base type at the corresponding site from the human genome reference;
(iv) identifying homozygous SNVs, diploid heterozygous SNVs, or non-diploid heterozygous SNVs from the SNVs identified in (iii), wherein
a homozygous SNV is define based on the percentage of sequence reads supporting the mutant base type different from the base type at the corresponding site from the human genome reference being 100%,
a diploid heterozygous SNV is define based on the percentage of sequence reads supporting the mutant base type different from the base type at the corresponding site from the human genome reference being no less than 25% and no large than 75%,
a non-diploid heterozygous SNV is define based on the percentage of sequence reads supporting the mutant base type different from the base type at the corresponding site from the human genome reference being less than 25% and larger than 0% or larger than 75% and less than 100%;
(v) determining a rate of homozygous SNVs, diploid heterozygous SNVs, or non-diploid heterozygous SNVs identified in (iv) for a window, wherein the rate of homozygous SNVs, diploid heterozygous SNVs, or non-diploid heterozygous SNVs represents the ratio of the number of homozygous SNVs, diploid heterozygous SNVs, or non-diploid heterozygous SNVs for the window to the average number of homozygous SNVs, diploid heterozygous SNVs, or non-diploid heterozygous SNVs among all windows in the biological sample; and
(vi) comparing the rate of homozygous SNVs, diploid heterozygous SNVs, or non-diploid heterozygous SNVs for individual windows determined from (v) with an average rate of homozygous SNVs, diploid heterozygous SNVs, or non-diploid heterozygous SNVs for corresponding individual windows established from a control population.
